(19) United States
(12) Patent Application Publication
(10) Pub. No.: US 2019/0125574 A1
(43) Pub. Date: May 2, 2019

(54) ADJUSTABLE BREATHING ASSISTANCE APPARATUS

(71) Applicant: Oventus Medical Limited, Indooroopilly, Queensland (AU)

(72) Inventors: Anthony IGNACIO, Bossley Park, New South Wales (AU); Benjamin LOW, Chapel Hill, Queensland (AU); Zoran MILIJASEVIC, Bayview, New South Wales (AU); Christopher Patrick HART, Eight Mile Plains, Queensland (AU); Neil ANDERSON, Roseville, New South Wales (AU); Michael Leigh SLATER, Southport, Queensland (AU)

(21) Appl. No.: 16/089,084

(22) PCT Filed: Mar. 29, 2017
(86) PCT No.: PCT/AU2017/050271

§ 371 Date: Sep. 27, 2018

(30) Foreign Application Priority Data
Mar. 30, 2016 (AU) 2016901171
Oct. 14, 2016 (CN) 2016211252195

(51) Int. Cl. A61F 5/56

(52) U.S. Cl. CPC A61F 5/566

(57) ABSTRACT

An apparatus for providing breathing assistance includes first and second bodies configured to be positioned within an oral cavity of a user. Each of the first and second bodies includes a recess configured to receive a respective set of teeth of a user. An adjustable mounting is configured to interconnect the first and second bodies to thereby allow a relative position of the first and second bodies to be adjusted.

### BACKGROUND OF THE INVENTION

The present invention relates to a breathing assistance apparatus and in particular an adjustable breathing assistance apparatus for adjusting the relative position of a user's jaws, for example to provide adjustable mandibular advancement.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Poor quality or ineffective breathing is an issue which can affect the performance of people in their day to day activities either while they are awake and/or when they are asleep. While awake this can be less optimal performance in activities such as sport or even while performing everyday tasks. While asleep breathing disorders can lead to snoring and/or sleep apnoea.

Snoring arises due to vibration of soft tissues within the respiratory pathways of an individual, and is typically caused by obstructed air movement during breathing while sleeping. Snoring can arise from a range of different physical causes such as blocked sinuses, and typically occurs when the muscles of the upper throat relax during sleep.

Snoring can also be associated with Obstructive Sleep Apnoea (OSA), which is caused by obstruction of the upper airway and results in repetitive pauses in breathing during normal sleep. Individuals having OSA often suffer from daytime sleepiness and fatigue associated with significant levels of sleep disturbance, whilst a partners sleep patterns are also often disturbed by associated snoring.

Current therapy for treatment of OSA can include lifestyle changes, the use of mechanical devices, such as oral or nasal devices that augment the airway, surgical procedures to enlarge and stabilize the airway during sleep, and continuous or variable positive airway pressure (CPAP, VPAP) devices.

However, surgical procedures can be severe and are not therefore widely used unless absolutely necessary. Whilst CPAP and VPAP devices have had a positive impact, these can be uncomfortable to wear for prolonged time periods, are expensive, and are often noisy, which can in turn lead to additional sleep disturbance. As a result, surgery, VPAP and CPAP treatment have limited application in treating sleep apnoea, and are not generally considered appropriate treatment for snoring.

In terms of other mechanical devices, nasal devices have been used that dilate the nasal airway using traction or splinting. However, these have typically not had much success and can be uncomfortable for a user.

US2004/194787 describes an anti-snoring device that includes a flexible hollow tube for insertion into the user's mouth, having proximal and distal ends and an outer perimeter. The tube includes an extra oral segment at its proximal end, an intraoral segment at its distal end and an intermediate segment extending therebetween. The extra oral and intraoral segments each include at least one opening. The extra oral segment is for extending beyond the user's outer lips, the intermediate segment is of a sufficient length for extending along the buccopharyngeal pathway of the user's mouth, and the intraoral segment is of a sufficient length for extending beyond a retromolar space in the user's mouth, into the oropharynx and terminating between the posterior tongue and the soft palate. The anti-snoring device also includes a stop extending from the outer perimeter of the tube on the intraoral segment for securing the intraoral segment within the user's oropharynx. However, whilst this arrangement can assist in providing an additional airway, and hence reduce snoring and apnoea events, it can be uncomfortable to wear and can move within the mouth during use, which can reduce device effectiveness and in turn lead to additional breathing problems.

US2005/150504 describes a device which is removably insertable in the mouth for facilitating breathing while sleeping which provides a clear unobstructed airway by protrusive positioning of the mandible and/or delivery of pressurized air to the back of the mouth. The device has upper and lower tooth-contacting members and an airway defined between them, and is designed specifically for use with CPAP machines. Consequently, this device can only be used in limited circumstances, where CPAP machines are available, and is only used in the treatment of sleep apnoea.

WO2012/155214 describes an apparatus for providing breathing assistance, the apparatus including a body including a recess for receiving teeth of a user to thereby position the body within an oral cavity of the user, a first opening extending beyond lips of a user to allow air from outside the oral cavity to be drawn in through the opening, a second opening provided in the oral cavity to allow air to be directed into a posterior region of the oral cavity and a channel connecting the first and second openings, the channel extending through at least part of a buccal sulcus of the user.

It is also known to provide mouth guards for use during sport. For example, US2013/0074851 describes a dental appliance including arms disposed about occlusal pad to secure the dental appliance in a removable fashion to the teeth of the user. The occlusal pad is formed from an occlusal pad material transformable between a pliable state and a non-pliable state, in various aspects. Associated methods of use are also disclosed herein.

US2013/0081640 describes an interchangeable mouth guard component system. The system includes a mouth guard base with a receiving recess positioned within at least a front surface of the base, a securely attachable mouth guard component positioned to fit within the recess, one or more attaching posts positioned on one of the recess or component and one or more holes positioned opposite the posts on either the recess or component. The mouth guard component, when attached, is positioned sufficient to maintain a substantially flush front surface of the base.

However, mouth guards for use in sport are not adapted to provide assistance with breathing and can in some circumstances make breathing more difficult.

WO2012/140021 describes a method for generating a virtual orthodontic element for use in manufacturing an orthodontic appliance for a patient. The method comprises obtaining a patient data set for said patient, the patient data set comprises a virtual 3D teeth model, where said virtual 3D teeth model comprises a virtual upper jaw and a virtual lower jaw resembling the upper jaw and lower jaw, respectively, of the patient's mouth, arranging the virtual upper jaw and the virtual lower jaw in an initial relative configuration in a virtual articulator which is able to simulate the articulation between the virtual upper jaw and the virtual lower jaw based at least on motion relative to at least one axis representing the terminal hinge axis of the patient, designing the virtual orthodontic element based on at least a part of the virtual 3D teeth model and the arrangement of the 3D teeth model in the virtual articulator.

However, use of the virtual articulation means that the resulting orthodontic element is not necessarily optimised for patient comfort as this may not take into account the particular articulation of the patient's jaws.

### SUMMARY OF THE PRESENT INVENTION

In one broad form an aspect of the present invention seeks to provide an apparatus for providing breathing assistance, the apparatus including: first and second bodies configured to be positioned within an oral cavity of a user, each of the first and second bodies including a recess configured to receive a respective set of teeth of a user in use; and, an adjustable mounting configured to interconnect the first and second bodies to thereby allow a relative position of the first and second bodies to be adjusted.

In one embodiment the adjustable mounting is configured to adjust a relative position of the first and second bodies in a longitudinal direction to thereby selectively advance the mandibular teeth relative to the maxillary teeth.

In one embodiment the longitudinal direction is aligned with a dental midline of the user in use.

In one embodiment the adjustable mounting is configured to allow at least one of: relative lateral movement of the first and second bodies; and, relative to and fro movement of the first and second bodies.

In one embodiment the adjustable mounting includes: a threaded shaft rotatably mounted to the first body, the shaft extending in a longitudinal direction; a threaded carriage mounted on the threaded shaft to allow the carriage to move longitudinally in response to rotation of the shaft; a peg coupled to the carriage; and, a slot in the second body, the peg being adapted to engage the slot in the second body.

In one embodiment the slot extends substantially in a lateral direction to allow relative lateral movement of the upper and lower parts.

In one embodiment the second body includes first and second slots spaced apart in a longitudinal direction, the peg being selectively coupled to one of the first and second slots to thereby adjust the relative position of the first and second bodies in the longitudinal direction.

In one embodiment the peg consists of a shaft having a shaft width and a head having a head width, the shaft width being smaller than the head width and wherein the slot is a keyhole slot having: a wide end defining an eyelet having an eyelet width greater than the head width, allowing the head to be received within the eyelet; and, a narrow end defined by a lip extending inwardly along part of a length of the slot, the narrow end having a slot width smaller than the head width but larger than the shaft width, allowing the peg to move therein whilst being retained by the head engaging an underside of the lip.

In one embodiment the keyhole slot has a slot depth greater than a head height of the peg, and wherein the lip has a lip height smaller than a shaft height of the shaft to allow the peg to move in the slot to allow relative to and fro movement of the first and second bodies.

In one embodiment the keyhole slot is defined by a slot component including: an arcuate elongate rear wall; end walls extending forwardly from proximate ends of the rear wall; a front rim coupled to the end walls and spaced from the rear wall so as to define a slot; and, an opening proximate one end of the rear wall to define the eyelet.

In one embodiment the threaded shaft is provided at least partially within the first opening and wherein the threaded shaft includes a plug at a first end, the plug being rotatably mounted within a socket in the first body and a head at a second end, the head being adapted to allow rotation of the threaded shaft.

In one embodiment the socket is defined by a socket component including: a socket body incorporating the socket; and, a flange extending around at least part of one edge of the socket body, wherein the socket component is mounted in a recessed opening within the first body so that the flange engages the recess with a flange surface being provided substantially flush with an underside of the first body.

In one embodiment the first body includes a collar extending at least partially around the threaded shaft proximate the second end, with the head being recessed within the collar.

In one embodiment the first body includes a carriage slot, the carriage being at least partially accommodated within the carriage slot allowing a position of the carriage relative to the first body to be determined.

In one embodiment the first body includes visual indicia, allowing a relative position of the first and second bodies in a longitudinal direction, to be determined.

In one embodiment the adjustable mounting includes: a first mounting member coupled to the first body; and, a second mounting member coupled to the second body, the second mounting member being slidably mounted to the first mounting member.

In one embodiment the adjustable mounting includes a locking member for locking the first and second mounting members in a fixed relative position.

In one embodiment the adjustable mounting includes an indicator that provides a visual indication of a relative position of the first and second mounting members.

In one embodiment the indicator includes a wheel rotatably mounted to one of the mounting members and adapted to rotate upon movement of the first and second mounting members.

In one embodiment the adjustable mounting includes an actuator for controlling a relative position of the first and second bodies.

In one embodiment the actuator includes a wheel rotatably mounted to one of the mounting members and adapted to engage the other mounting member to thereby cause relative movement of the mounting members upon rotation of the wheel.

In one embodiment the first mounting member defines a sleeve and the second mounting member is slidably mounted within the sleeve.

In one embodiment the adjustable mounting includes: wings extending from the first body towards the second body in use; blocks adjustably mounted to the second body to allow the blocks to be moved in a longitudinal direction and wherein in use the wings engage the blocks to constrain relative longitudinal movement of the first and second bodies to thereby selectively advance the mandibular teeth relative to the maxillary teeth.

In one embodiment a forward surface of the wings and a rearward surface of the block are sloped to thereby urge the second body in a longitudinal forward direction as the first and second bodies are brought into engagement.

In one embodiment the adjustable mounting includes arms pivotally mounted to the first and second bodies to hold the bodies in a defined relative longitudinal position.

In one embodiment each arm is mounted to a fore side wall of the first body and a rear side wall of the second body.

In one embodiment the mounting is adjusted by at least one of: adjusting a length of the arms; and, adjusting a pivot point.

In one embodiment at least one of the first and second bodies includes: at least one first opening for allowing airflow between lips of the user; at least one second opening provided in the oral cavity to allow air flow into and out of a posterior region of the oral cavity; and, at least one channel connecting the at least one first and second openings.

In one embodiment each channel includes: a first channel portion extending through the user's buccal cavity; and, a second channel portion in fluid communication with the first channel portion and extending between the user's maxillary and mandibular teeth.

In one embodiment at least one of the cross sectional shape and cross sectional area of at least one of the first and second channel portions varies from the first opening to the second opening.

In one embodiment a shape and size of the channels varies in accordance with an anatomy of the oral cavity of the user.

In one embodiment each channel directs air through a hammular notch of the user.

In one embodiment the second openings are angled inwardly at least one of: between 10° and 50°; between 20° and 40°; and, approximately 30°.

In one embodiment the second openings are positioned over a last or back tooth on each side of the top jaw.

In one embodiment each body is made of at least one of: nylon; metal; titanium alloys; high strength polymers; and, cobalt chromium alloys.

In one embodiment each body is made using at least one of: additive manufacturing; selective laser sintering; and, moulding.

In one embodiment the apparatus is made by selecting one of a plurality of fixed size first bodies and one of a plurality of fixed size second bodies, based on a shape of jaws and teeth of the user.

In one embodiment each body is coated with at least one of: a medical grade polymer; a medical grade elastomer; silicone; polyurethane; epoxy; and, parylene.

In one embodiment at least part of each body is polished using at least one of mechanical and electrochemical polishing.

In one embodiment the apparatus includes at least one insert positioned in a recess of one of the first and second bodies, the insert being positioned at least partially between the user's teeth and the respective body in use.

In one embodiment the insert is customised for a user's teeth.

In one embodiment the insert is at least one of removable and replaceable.

In one embodiment the apparatus can be used with a plurality of different inserts.

In one embodiment the insert is at least one of: a temporary insert; and, a semi-permanent insert.

In one embodiment the temporary insert is at least one of: a moulded insert; a silicone moulded insert; and, created at least partially in situ.

In one embodiment the semi-permanent insert is at least one of: a moulded insert; an acrylic insert; and, created at least in part using additive manufacturing.

In one embodiment the insert is made of at least one of: metals; ceramics; a polymer; polyvinylsiloxane; polyurethane; and, ethylvinylacetate.

In one embodiment each insert includes a base attached to the insert by a number of pillars and wherein, in use, the base is positioned on an underside of the body, with the pillars extending through apertures in the body to thereby secure the insert to the body.

In one embodiment each recess includes spaced apart recess side walls extending from a recess base, and wherein each insert includes an insert body shaped to at least partially conform to the recess side walls and recess base of a respective recess.

In one embodiment at least one recess side wall includes a recess lip projecting inwardly from at least part of the side wall, and wherein the recess lip is adapted to engage at least part of the insert body to thereby at least partially retain the insert body within the recess.

In one embodiment the recess lip extends inwardly from each recess side wall.

In one embodiment the recess lip extends inwardly from a distal edge of each recess side wall.

In one embodiment each insert includes a step in an outer surface of the insert and wherein the step and recess lip engage to thereby retain the insert in the recess.

In one embodiment the first body includes a base and a cover coupled to the base, the base and cover defining: at least one first opening for allowing airflow between lips of the user; at least one second opening provided in the oral cavity to allow air flow into and out of a posterior region of the oral cavity; and, at least one channel connecting the at least one first and second openings.

In one broad form an aspect of the present invention seeks to provide apparatus for providing breathing assistance, the appliance including: first and second bodies configured to be positioned within an oral cavity of a user, each of the first and second bodies including a recess having spaced apart recess side walls extending from a recess base and a recess lip projecting inwardly from at least part of at least one side wall; and, first and second inserts, each insert having an insert body shaped to at least partially conform to the recess side walls and recess base of a respective recess, the insert body and recess lip engaging to thereby retain the insert within the respective recess, and the insert body being configured to receive a respective set of teeth of a user in use.

In one broad form an aspect of the present invention seeks to provide a method for providing an apparatus for providing breathing assistance, the method including: determining a shape of a user's upper and lower jaws; selecting one of a plurality of standard sized first and second bodies based on the determined shape of the upper and lower jaws respectively, each of the first and second bodies including a recess configured to receive a respective set of teeth of a user in use; and, creating first and second inserts at least in part by moulding material to the shape of the user's mandibular and maxillary teeth, each insert having an insert body shaped to at least partially conform to the recess side walls and recess base of a respective recess, and being configured to receive a respective set of teeth of a user in use.

In one broad form an aspect of the present invention seeks to provide apparatus for adjusting relative jaw position, the apparatus including: first and second bodies configured to be positioned within an oral cavity of a user, each of the first and second bodies including a recess configured to receive a respective set of teeth of a user in use; and, an adjustable mounting configured to interconnect the first and second bodies to thereby allow a relative position of the first and second bodies to be adjusted.

In one embodiment the adjustable mounting includes: a first mounting member coupled to the first body; and, a second mounting member coupled to the second body, the second mounting member being slidably mounted to the first mounting member.

In one embodiment the adjustable mounting includes a locking member for locking the first and second mounting members in a fixed relative position.

In one embodiment the adjustable mounting includes an indicator that provides a visual indication of a relative position of the first and second mounting members.

In one embodiment the indicator includes a wheel rotatably mounted to one of the mounting members and adapted to rotate upon movement of the first and second mounting members.

In one embodiment the adjustable mounting includes an actuator for controlling a relative position of the first and second bodies.

In one embodiment the actuator includes a wheel rotatably mounted to one of the mounting members and adapted to engage the other mounting member to thereby cause relative movement of the mounting members upon rotation of the wheel.

In one embodiment the first mounting member defines a sleeve and the second mounting member is slidably mounted within the sleeve.

In one broad form an aspect of the present invention seeks to provide an apparatus for use in creating moulded inserts for use in an oral appliance, the apparatus including: first and second bodies configured to be positioned within an oral cavity of a user, each of the first and second bodies including a recess configured to receive a curable mould material, which is moulded to a respective set of teeth of a user when a user bites into the mould material in use; and, an adjustable mounting configured to interconnect the first and second bodies to thereby allow a relative position of the first and second bodies to be adjusted to thereby allow jaws of the user to be relatively positioned when the user is biting into the mould material.

In one broad form an aspect of the present invention seeks to provide a method for use in creating moulded inserts for use in an oral appliance, the method including: providing first and second bodies interconnected via an adjustable mounting configured to interconnect the first and second bodies to thereby allow a relative position of the first and second bodies to be adjusted, each of the first and second bodies including a recess; filling the recess with a mould material; inserting the bodies into the oral cavity of the user, with the user biting into the mould material; relatively positioning the first and second bodies using the adjustable mounting to achieve a desired relative position of jaws of the user while the user is biting into the mould material; and, curing the mould material, the cured mould material being used to provide inserts for use in an oral appliance.

In one broad form an aspect of the present invention seeks to provide an apparatus for providing breathing assistance, the apparatus including: a first body including a base and a cover coupled to the base, the base and cover define: at least one first opening for allowing airflow between lips of the user; at least one second opening provided in the oral cavity to allow air flow into and out of a posterior region of the oral cavity; and, at least one channel connecting the at least one first and second openings; a second body configured to be positioned within an oral cavity of a user; and, first and second inserts, each being coupled to a respective one of the first and second bodies, each insert being positioned at least partially between the user's teeth and the respective body in use, and the inserts being moulded to fit the user's teeth.

In one embodiment the second openings are at least one of: angled inwardly at least one of: between 10° and 50°; between 20° and 40°; and, approximately 30°; positioned over a last or back tooth on each side of the top jaw; and, configured to direct air through a hammular notch of the user.

In one embodiment each body is made of an injection moulded polymer that is at least one of: a thermosetting polymer; a thermoplastic polymer; silicone; an elastomer; polycarbonate; acrylonitrile butadiene styrene; polyvinylsiloxane; polyurethane; and, ethylvinylacetate.

In one embodiment each insert is made of at least one of: an injection moulded polymer; an additive printed material; and, a deformable material set in the shape of the user's teeth.

In one embodiment the first body includes an adjustable mounting configured to interconnect the first and second bodies to thereby allow a relative position of the first and second bodies to be adjusted.

In one embodiment the adjustable mounting is configured to adjust a relative position of the first and second bodies in a longitudinal direction aligned with a dental midline of the user in use to thereby selectively advance the mandibular teeth relative to the maxillary teeth.

In one embodiment the adjustable mounting is configured to allow at least one of: relative lateral movement of the first and second bodies; and, relative to and fro movement of the first and second bodies.

In one embodiment the adjustable mounting includes: a threaded shaft rotatably mounted to the first body, the shaft extending in a longitudinal direction; a threaded carriage mounted on the threaded shaft to allow the carriage to move longitudinally in response to rotation of the shaft; a peg coupled to the carriage; and, a slot in the second body, the peg being adapted to engage the slot in the second body and wherein the slot extends substantially in a lateral direction to allow relative lateral movement of the upper and lower parts.

In one embodiment the threaded shaft is provided at least partially within the first opening and includes: a plug at a first end, the plug being rotatably mounted within a socket in the first body; and, a head at a second end, the head being retained in a collar extending at least partially around the threaded shaft proximate the second end, with the head being recessed within the collar and being adapted to allow rotation of the threaded shaft.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

An example of the present invention will now be described with reference to the accompanying drawings, in which:—

FIG. 1A is a schematic perspective top side view of an apparatus for providing breathing assistance;

FIG. 1B is a schematic perspective underside view of the apparatus of FIG. 1A;

FIG. 1C is a schematic front view of the apparatus of FIG. 1A;

FIG. 1D is a schematic rear view of the apparatus of FIG. 1A;

FIG. 1E is a schematic side view of the apparatus of FIG. 1A with the second body in a rearward position;

FIG. 1F is a schematic side view of the apparatus of FIG. 1A with the second body in a forward position;

FIG. 2A is a schematic perspective top side view of an example of a first body;

FIG. 2B is a schematic perspective underside view of the first body of FIG. 2A;

FIG. 2C is a schematic cross sectional view of the first body of FIG. 2A when viewed along the line A-A′ of FIG. 1D;

FIG. 3A is a schematic underside perspective view of the adjustable mounting in a retracted position to support the second body in the rearward position;

FIG. 3B is a schematic underside perspective view of the adjustable mounting in a forward position to support the second body in the forward position;

FIG. 3C is a schematic front perspective view of the body of FIG. 2A and the adjustable mounting of FIG. 3A with the adjustable mounting in a forward position;

FIG. 3D is a schematic underside perspective view of the body of FIG. 2A and the adjustable mounting of FIG. 3A with the adjustable mounting in a forward position;

FIG. 4A is a schematic perspective view of a first example of the second body;

FIG. 4B is a schematic perspective underside view of the second body of FIG. 4A;

FIG. 4C is a schematic plan close-up view of the keyhole slot of the second body of FIG. 4A;

FIG. 4D is a schematic close-up cross-sectional view of the keyhole slot of the second body of FIG. 4A when viewed along the line A-A′ of FIG. 1D;

FIG. 4E is a schematic close-up cross-sectional view of an alternative slot and peg design in an engaged configuration;

FIG. 4F is a schematic close-up cross-sectional view of an alternative slot and peg design in a disengaged configuration;

FIG. 5A is a schematic close-up perspective underside view of the first and second bodies;

FIG. 5B is a schematic close-up cross-sectional view of the first and second bodies, where the second body is in a rearward position when viewed along the line A-A′ of FIG. 1D;

FIG. 5C is a schematic close-up cross-sectional view of the first and second bodies, where the second body is in a forward position when viewed along the line A-A′ of FIG. 1D;

FIG. 6A is a schematic perspective view of a second example of a second body;

FIG. 6B is a schematic perspective view of an apparatus for providing breathing assistance incorporating the second body of FIG. 6A;

FIG. 7A is a schematic perspective top side view of an apparatus for adjusting a user's jaw position;

FIG. 7B is a schematic perspective underside view of the apparatus of FIG. 7A;

FIG. 7C is a schematic side view of the apparatus of FIG. 7A;

FIG. 7D is a schematic plan view of the apparatus of FIG. 7A;

FIG. 7E is a schematic side cross-sectional view of the apparatus of FIG. 7A;

FIG. 7F is a schematic perspective exploded view of the apparatus of FIG. 7A;

FIG. 8A is a schematic perspective top side view of an example of an alternative adjustable mounting;

FIG. 8B is a schematic perspective underside view of the adjustable mounting of FIG. 8A;

FIG. 8C is a schematic perspective top side rear view of the adjustable mounting of FIG. 8A;

FIG. 8D is a schematic perspective underside view of the adjustable mounting in a first body;

FIG. 8E is a schematic perspective top side view of the slot body in a second body;

FIG. 9 is a flow chart of an example of a process for fitting an apparatus for providing breathing assistance;

FIG. 10A is a schematic perspective top side view of a further example of an apparatus for providing breathing assistance;

FIG. 10B is a schematic perspective underside view of the apparatus of FIG. 10A;

FIG. 10C is a schematic front view of the apparatus of FIG. 10A;

FIG. 10D is a schematic rear view of the apparatus of FIG. 10A;

FIG. 10E is a schematic side view of the apparatus of FIG. 10A with the second body in a rearward position; and,

FIG. 10F is a schematic side view of the apparatus of FIG. 10A with the second body in a forward position;

FIG. 11A is a schematic top side exploded perspective view of the first body and insert of FIG. 10A;

FIG. 11B is a schematic underside exploded perspective view of the first body and insert of FIG. 10A;

FIG. 11C is a schematic perspective top side view of an example of a first body;

FIG. 11D is a schematic perspective underside view of the first body of FIG. 11A;

FIG. 11E is a schematic cross sectional view of the first body of FIG. 11A;

FIG. 12A is a schematic top side exploded perspective view of the second body and insert of FIG. 10A;

FIG. 12B is a schematic underside exploded perspective view of the second body and insert of FIG. 10A;

FIG. 12C is a schematic perspective view of a first example of the second body;

FIG. 12D is a schematic perspective underside view of the second body of FIG. 12A;

FIG. 12E is a schematic plan close-up view of the keyhole slot of the second body of FIG. 12A;

FIG. 12F is a schematic cross-sectional view of the second body and insert of FIG. 12A;

FIG. 13A is a schematic rear top side perspective view of an alternative example of a second body and insert of FIG. 10A;

FIG. 13B is a schematic rear top side perspective view of the insert of FIG. 13A;

FIG. 13C is a schematic rear top side perspective view of the first body of FIG. 13A;

FIG. 13D is a first schematic rear top side cut-away perspective view of the first body and insert of FIG. 13A;

FIG. 13E is a second schematic rear top side cut-away perspective view of the first body and insert of FIG. 13A;

FIG. 14A is a schematic perspective top front side view of a further example of an apparatus for providing breathing assistance;

FIG. 14B is a schematic side view of the apparatus of FIG. 14A;

FIG. 14C is a schematic perspective rear underside view of the apparatus of FIG. 14A;

FIG. 14D is a schematic perspective top rear side view of the second body of FIG. 14A;

FIG. 14E is a schematic top cut-away view of the apparatus of FIG. 14A;

FIG. 14F is a schematic rear cut-away view of the apparatus of FIG. 14A;

FIG. 15A is a schematic front top side perspective view of an alternative example of an apparatus for providing breathing assistance;

FIG. 15B is a schematic rear top side perspective view of the apparatus of FIG. 15A;

FIG. 15C is a schematic side view of the apparatus of FIG. 15A; and,

FIG. 15D is a schematic front view of the apparatus of FIG. 15A.

### DETAILED DESCRIPTION OF THE PREFERRED EMBODIMENTS

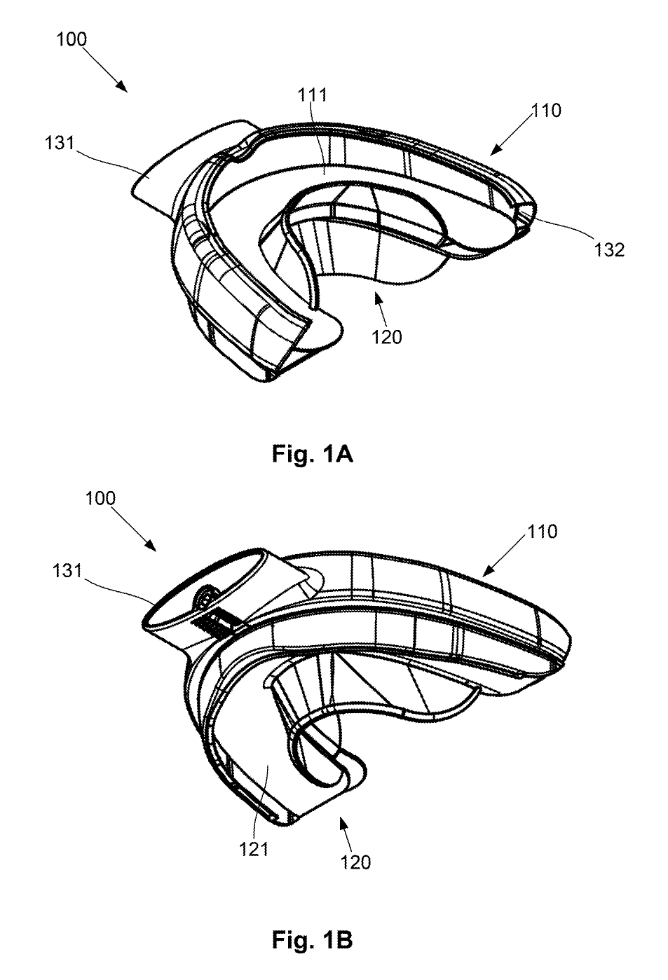

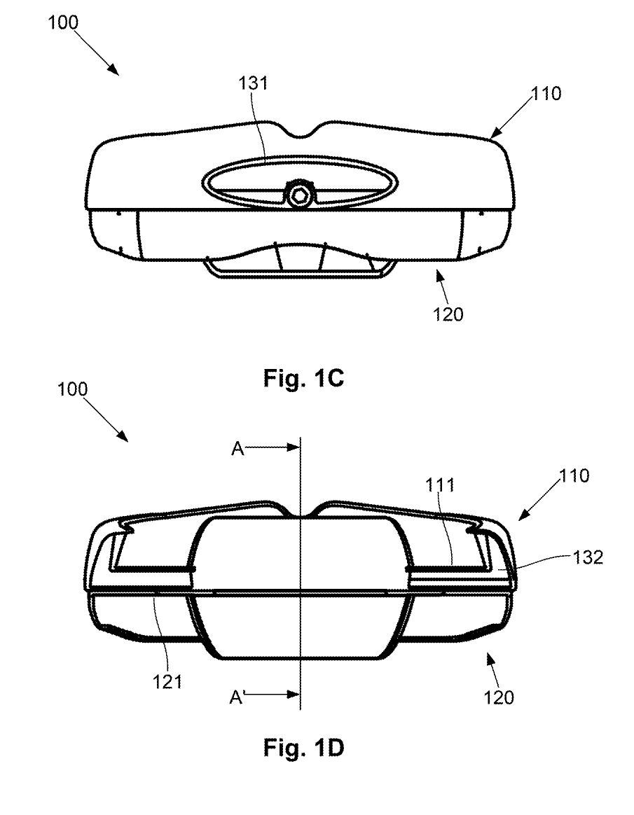

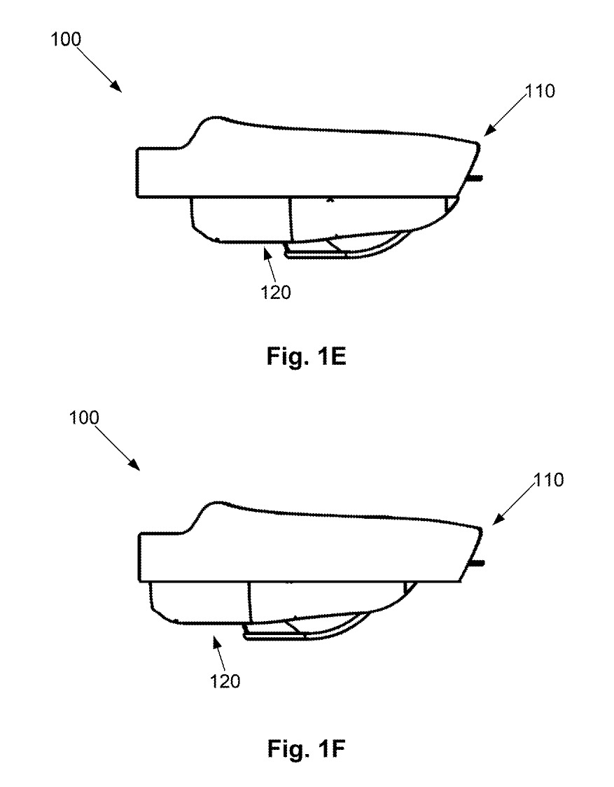

An example of an apparatus for providing breathing assistance will now be described with reference to FIGS. 1A to 1F.

In this example, the apparatus 100 includes first and second bodies 110, 120 configured to be positioned within an oral cavity of a user. Each of the first and second bodies 110, 120 includes a recess 111, 121 configured to receive a respective set of teeth of a user in use. The teeth may be received directly in the recess, or alternatively can be received in an insert positioned within the recess, as will be described in more detail below.

The apparatus further includes an adjustable mounting configured to interconnect the first and second bodies to allow a relative position of the first and second bodies to be adjusted. The form of the adjustable mounting will vary depending upon the performed implementation and specific example will be described in more detail below.

Thus, the apparatus provides an oral appliance for providing breathing assistance. This can be used during sleep, for example for the treatment of both snoring and sleep apnoea, and can also be used at other times, for example in the treatment of respiratory conditions, such as emphysema, and to assist in jaw placement to provide an adequate airway, for use during surgery, CPR (Cardiopulmonary resuscitation), or the like.

As respective sets of teeth of the user are provided in the first and second body recesses, adjusting the relative position of the first and second bodies can be used to selectively position the mandibular teeth relative to the user's maxillary teeth, for example to provide mandibular advancement. In this regard, it is known that mandibular advancement can assist in holding open the user's airway, which in turn can reduce snoring. For example, temporomandibular joint disorder (TMD) arises when the upper and lower jaws are misaligned. This may be naturally occurring or can result from injury, or the like. Regardless, such jaw misalignment tends to contribute to airway obstructions by changing the shape of the upper airway, and moving the tongue towards the posterior of the oral cavity, which can in turn exacerbate issues associated with OSA and snoring. Accordingly, by allowing the relative position of the first and second bodies to be adjusted, this allows the jaws of the user to be aligned thereby reducing the effects of TMD, and hence further reducing the likelihood of snoring and OSA.

Accordingly, the provision of the adjustable mounting, allowing the relative position of the first and second bodies to be controllably adjusted, in turn allows a relative position of the lower jaw to be adjusted, for example to provide for mandibular advancement. This can assist significantly in the reduction of snoring.

Furthermore, typically at least one of the first and second bodies 110, 120 includes at least one first opening 131 for allowing airflow between the lips of the user, and at least one second opening 132 provided in the oral cavity of the user, the first and second openings being interconnected via one or more channels (not shown) to allow airflow into and out of a posterior region of the oral cavity.

Thus, the device has a first opening 131, which can extend beyond the lips, or at least keep the lips apart, to allow airflow therethrough. Air passes through airways defined by the channels, and is directed into a posterior region of the mouth through second openings 132, bypassing issues with tongue and lower jaw position. In more severe cases, the first opening 131 can be connected to an external device, such as a CPAP (Continuous Positive Airway Pressure) machine, air supply, or the like providing more comfort and increased patient compliance compared to a mask. In such situations, the airways 131 and 132 may be divided into two or more airwaves working together or in opposite directions.

Providing air flow directly into a posterior portion of the user's oral cavity has a number of benefits. In particular, this avoids obstructions created by the nasal cavity, soft palate and tongue which can lead to snoring and apnoea events, and helps reduce the drying effects of air flow, which can in turn lead to user discomfort. This makes the apparatus comfortable to wear whilst ensuring an unobstructed air flow thereby preventing snoring and apnoea events. Thus, for example, nasal obstructions can be bypassed by air flow through the apparatus, thereby bypassing the nasal airway or adding to it in the case of a partial obstruction. Furthermore, air flowing below or on both sides of the soft palette helps prevent collapse of the soft palate, which can in turn lead to additional obstruction.

Accordingly, it will be appreciated that the combination of the airway and the ability to adjust the relative position of the first and second bodies can provide assistance beyond that afforded by either the airway or mandibular advancement alone. However, it will also be appreciated from examples described in more detail below that the airway is not necessarily required depending on the intended usage of the arrangement. In this regard, a similar configuration of adjustable bodies can be used for the purpose of adjusting a relative position of the user's jaws, for example to correct muscular-skeletal defects and/or for use in creating moulded inserts for use with an oral appliance, as will be described in more detail below.

A number of further possible features will now be described.

In one example, the apparatus allows a relative position of the first and second bodies to be adjusted in a longitudinal direction, with the second body being positionable at points between rearward and forward positions shown in FIGS. 1E and 1F respectively. In this regard the longitudinal direction is typically aligned with a dental midline of the user in use, so that this configuration can provide mandibular advancement, although it will be appreciated that other forms of adjustment could be provided.

In the current example, the first opening, second opening and channel are provided in the first body 110, with the mandibular teeth being received by the second body, so that as the position of the second body relative to the first is adjusted, the user's lower jaw is positioned with respect to the upper jaw. However, it will be appreciated that this is not necessarily essential and alternatively the first and second openings and channel could be provided in the second body.

The adjustable mounting is typically configured to further allow relative lateral movement of the first and second bodies, and to allow relative to-and-fro movement of the first and second bodies. These movements can be largely unconstrained, allowing a user to move their jaw laterally, or to open and close their jaws, whilst remaining engaged with the apparatus. This can aid comfort when using the device, whilst ensuring mandibular advancement is maintained.

The adjustable mounting can be of any appropriate form. In one example, this includes a threaded shaft rotatably mounted to the first body, arranged to extend in a longitudinal direction and which has a threaded carriage mounted thereon to allow the carriage to move longitudinally in response to rotation of the shaft. The carriage supports a peg, which in use engages a slot in the second body, allowing the second body to move longitudinally as the shaft is rotated.

In one example, the peg consists of a shaft having a shaft width and a head having a head width, the shaft width being smaller than the head width. In this case, the slot can be formed from a keyhole slot having a wide end defining an eyelet having an eyelet width greater than the head width, allowing the head to be received within the eyelet and a narrow end defined by a lip extending inwardly along part of a length of the slot, the narrow end having a slot width smaller than the head width but larger than the shaft width, allowing the peg to move therein whilst being retained by the head engaging an underside of the lip. In particular, by having the slot extend laterally, this allows for relative unconstrained lateral movement of the first and second bodies.

Additionally, the keyhole slot can have a slot depth greater than a head height of the peg, and wherein the lip has a lip height smaller than a shaft height of the shaft to allow the peg to move in the slot to allow for relative to and fro movement of the first and second bodies. However, this is not essential, and in other examples, to and fro movement of the first and second bodies could be constrained. Additionally, in one example, the adjustment mechanism could be configured to engage in a direction perpendicular to a plane of the first and second bodies, thereby effectively locking the first and second bodies and preventing these being inadvertently separated in use.

In one example, the second body can also include first and second slots spaced apart in a longitudinal direction, allowing the peg to be selectively coupled to one of the first and second slots. This can be used to provide coarse adjustment of the relative longitudinal position of the first and second bodies, with fine adjustment being achieved through rotation of the shaft.

In one example, the threaded shaft is mounted at least partially within the first opening in the first body. To achieve this, the threaded shaft can include a plug that is rotatably mounted within a corresponding socket in the first body, whilst the threaded shaft includes a head at a second end, which allows the shaft to be rotated, for example through engagement with a tool such as a hex key or similar. The first body typically also includes a collar extending at least partially around the threaded shaft approximate the second end with the head being recessed within the collar, to thereby hold the second end in position, whilst allowing rotation of the head end.

Typically, the first body can include a carriage slot with the carriage being at least partially accommodated within the carriage slot allowing a position of the carriage relative to the first body to be determined. This can be assisted by the use of visual indicia, such as markings provided on an outer surface of the first opening, allowing a position of the carriage relative to the first body to be measured, so a user can move the first and second bodies to specific positions.

It will be appreciated however, that alternative forms of adjustable mounting could be used and that the above described example is for the purpose of illustration only.

For example, the adjustable mounting could include any linearly movable mounting, such as a sliding arrangement that allows the first and second bodies to slide relative to each. The bodies could then be held in a desired position using any suitable mechanism, such as a locking mechanism, frictional engagement or the like.

In one example, the mounting includes a first mounting member coupled to the first body and a second mounting member coupled to the second body, the second mounting member being slidably mounted to the first mounting member. However, this is not essential and alternatively the slidable engagement could be achieved through appropriate configuration of the first and second bodies themselves.

In one example, the adjustable mounting includes a locking member for locking the first and second mounting members in a fixed relative position. The locking member could be of any form, and could for example include a fastener or locking bar mounted to one of the first and second bodies, and which selectively engages the other body, either via frictional engagement, mechanical coupling or the like, to thereby lock the other body in position.

The adjustable mounting can include an indicator that provides a visual indication of a relative position of the first and second mounting members. This could include a wheel rotatably mounted to one of the mounting members and adapted to rotate upon movement of the first and second mounting members.

In one example, the apparatus includes an actuator for controlling a relative position of the first and second bodies, which could include a wheel rotatably mounted to one of the mounting members and adapted to engage the other mounting member to thereby cause relative movement of the mounting members upon rotation of the wheel. Thus, it will be appreciated from this that in one example, the actuator and indicator could be a single component serving a dual purpose, but this is not essential.

In one example, the first mounting member defines a sleeve, with the second mounting member being slidably mounted within the sleeve, although this is not essential and other arrangements, such as the use of a track and sliding body could be used.

When an airway is provided, the channels can have a wide variety of configurations and may be sized and shaped depending on the anatomy of the oral cavity of the user. This is typically done to maximise the available airway, whilst ensuring comfort for the user. In one example, the total airway has a cross sectional area of at least 50 mm², at least 70 mm², at least 90 mm², at least 100 mm² and at least 110 mm². The dimensions selected will vary depending on a wide range of factors, including whether the device is required to provide a partial or complete airway, for example to bypass a partial or complete blockage. Additionally, this will depend on the intended use and the associated airflow requirements. Typically the dimensions of the channels and/or openings 131, 132 are selected so that in conjunction with the user's existing airways, the total airway available corresponds to the cross sectional area of an airway in a healthy subject for both nasal and pharyngeal airways. In any event, the cross sectional areas used will depend on the preferred implementation and intended use, so for example, a smaller cross sectional area may be used for children, adolescents, or individuals with only partial obstructions. In contrast increased cross sectional areas may be used where a high flow rate is required, for example in the event that the device is to be used to provide breathing assistance during exercise.

In one example, each channel includes a portion extending through the buccal cavity, between the user's cheeks and teeth, and a portion extending between the user's maxillary and mandibular teeth. This arrangement maximises the cross sectional area of the channels, whilst maintaining comfort for the user, by distributing the airway between the user's teeth and cheeks, and between the user's teeth.

The cross sectional area of the first and second channel portions can vary between the first and second openings, allowing the overall cross section of the channel to be maintained, whilst having the channel conform as far as possible to the natural space available in the oral cavity. It will be appreciated that any variation can be used, depending for example on the configuration of the user's oral cavity. Thus, the airways defined by the channels have a cross section that is shaped to conform to a person's oral cavity, and in particular the available space between the maxillary and mandibular teeth, as well as between the teeth and the cheeks.

In one example, the second openings are angled inwardly at between 10° and 50°, more typically between 20° and 40° and preferably about 30° to assist in airflow into and out of the oral cavity, and in particular to direct airflow towards the centre of the oral cavity. Additionally and/or alternatively, the second openings are positioned over the last or back tooth on each side of the top jaw.

In one example, the bodies are manufactured using additive manufacturing, such as a 3D printing process. This is particularly beneficial as it allows the channel arrangement to be made, whilst minimising the thickness of the channel walls. This helps maximise the cross sectional area of the channels, thereby assisting airflow, whilst minimising overall device volume, thereby helping to maintain comfort. For example, the use of additive manufacturing allows a body having channel wall thicknesses of less than 0.5 mm and more typically approximately 0.3 mm or less, although it will be appreciated that other thicknesses can be accommodated if required. Thus, this significantly reduces the volume/bulk compared to an acrylic device made using standard processes, thereby maximising the available airway size, whilst also leading to more user comfort and improved compliance.

In one example, the bodies 110 are made of metal and in particular a titanium alloy and/or cobalt chromium alloy, although it will be appreciated that any suitable material may be used, including high strength polymers, plastics, VeroGlaze (MED620) dental material, or the like. This can be achieved using additive printing, injection moulding or any other suitable technique. The bodies can be coated with a medical grade polymer and in one example, a medical grade elastomer, such as silicone or polyurethane, epoxy or parylene, for improved comfort as well as ensuring biocompatability. In one example, the coating can include an Active Composite Guidance, which is a 3 dimensional composite resin with different shapes and sizes and which can be bonded to the body to ensure accurate positioning of the body with respect to the user's teeth. Coatings can be applied to the body using any suitable technique, such as dip coating, vapour coating, or spray coating the body, thereby ensuring all exposed surfaces, including internal surfaces of the channels, are coated. As part of this process, this can include applying primers to the body prior to coating, thereby ensuring the coating adheres to the body. As an alternative, or in addition to coating, at least part of the body can be polished using at least one of mechanical and electrochemical polishing.

In one example, the bodies are custom made by measuring the oral cavity of the user, for example by taking dental impressions, a series of photos, or scans of the user's teeth and/or oral cavity and then customising the apparatus based on the measured size, as will be described in more detail below.

However, more typically a range of standard sizes of first and second bodies can be produced, with an appropriate first and second body being selected based on a closest fit to the intended user. Custom fitting can then be achieved using inserts positioned in the recesses of the first and second bodies and positioned between the user's teeth and the respective body in use. Each insert is typically customised for a user's teeth and is adapted to be removable and/or replaceable.

It will be appreciated that this provides a straightforward mechanism for providing an apparatus for providing breathing assistance. In this case, the method includes determining a shape of a user's upper and lower jaws, selecting one of a plurality of standard sized first and second bodies based on the determined shape of the upper and lower jaws respectively, each of the first and second bodies including a recess configured to receive a respective set of teeth of a user in use and then creating first and second inserts at least in part by moulding material to the shape of the user's mandibular and maxillary teeth, each insert having an insert body shaped to at least partially conform to the recess side walls and recess base of a respective recess, and being configured to receive a respective set of teeth of a user in use.

In one example, the apparatus can be used be with a plurality of different inserts, which can be used for example to provide different levels of fit, comfort, support or the like. The inserts can also be either temporary or semi-permanent, and may be made from different materials depending on their intended use. For example, a temporary insert could be created upon the initial fitting of a breathing assist apparatus, using a silicone which is moulded in-situ, with this being replaced by a subsequent semi-permanent insert, such as a 3D printed acrylic insert, once there has been opportunity for this to be manufactured. This allows an initial fitting to be performed when the apparatus is initially supplied with temporary inserts, with semi-permanent acrylic inserts being subsequently manufactured and provided to the user once ready.

Whilst the inserts could be fitted using any suitable technique, in one example, each recess includes spaced apart recess side walls extending upwardly from a recess base, with the insert having an insert body shaped to at least partially conform to the recess side walls and base. This allows the insert to be provided in the recess, so it does not move laterally or longitudinally within the recess. Whilst the insert could then be held in place using bonding such as adhesives, fasteners, or the like, in one example this is achieved using mechanical engagement between the insert and recess. In particular, the recess side wall can include a recess lip projecting inwardly from at least part of the side wall, and more typically a distal edge of the side wall, with the lip engaging at least part of the insert body, such as a step or shoulder provided on the body, to thereby retain the insert body within the recess. The recess lips can extend along an entire length of each side wall, although this is not essential and other configurations, such as having lips on one side wall per recess, or extending along one or more different parts of the side wall could be used.

It will be appreciated that the use of mechanical engagement to retain an insert in position could also be performed on breathing appliances formed from a single unitary body. Thus, in one example, an oral appliance for providing breathing assistance includes a body configured to be positioned within an oral cavity of a user, the body including first and second recesses having spaced apart recess side walls extending from a recess base and a recess lip projecting inwardly from at least part of at least one side wall. The apparatus further includes first and second inserts, each insert having an insert body shaped to at least partially conform to the recess side walls and recess base of a respective recess, the insert body and recess lip engaging to thereby retain the insert within the respective recess, and the insert body being configured to receive a respective set of teeth of a user in use.

In one example, the apparatus can be used for adjusting a relative jaw position of the user by providing first and second bodies configured to be positioned within an oral cavity of a user, each of the first and second bodies including a recess configured to receive a respective set of teeth of a user in use and an adjustable mounting configured to interconnect the first and second bodies to thereby allow a relative position of the first and second bodies to be adjusted.

In another example, the apparatus can be used for creating inserts for use in an oral appliance of the user by providing first and second bodies configured to be positioned within an oral cavity of a user, each of the first and second bodies including a recess configured to receive a curable mould material, which is moulded to a respective set of teeth of a user when a user bites into the mould material in use and an adjustable mounting configured to interconnect the first and second bodies to thereby allow a relative position of the first and second bodies to be adjusted to thereby allow jaws of the user to be relatively positioned when the user is biting into the mould material.

In this example, the process for creating an insert can include providing the first and second bodies, filling each recess with a mould material, inserting the bodies into the oral cavity of the user, with the user biting into the mould material, relatively positioning the first and second bodies using the adjustable mounting to achieve a desired relative position of jaws of the user while the user is biting into the mould material and, curing the mould material, the cured mould material being used to provide inserts for use in an oral appliance.

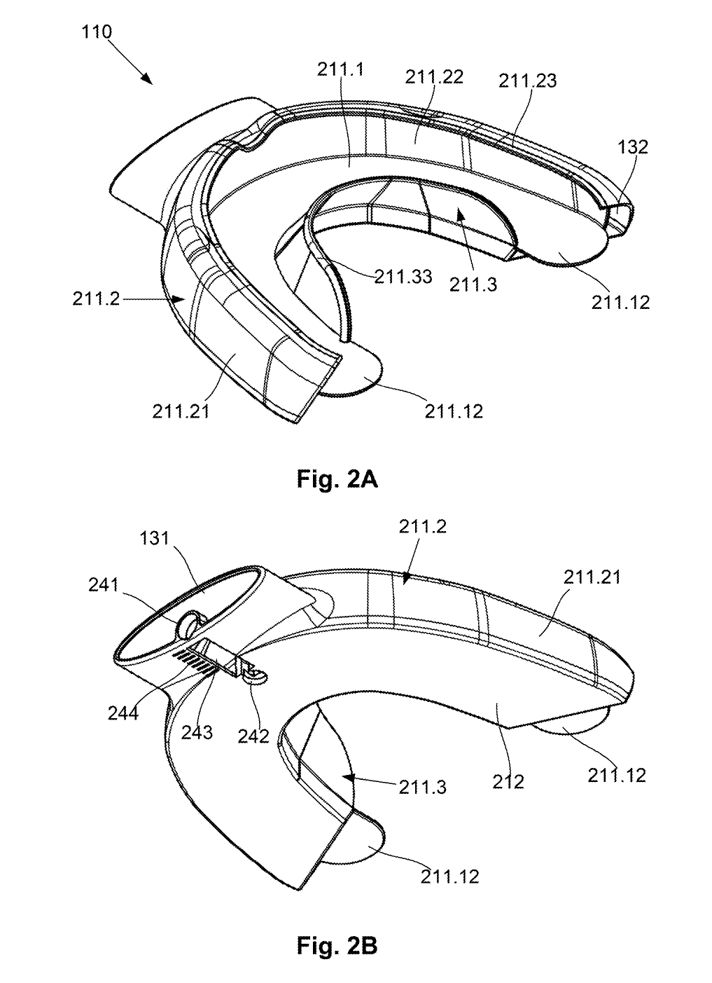

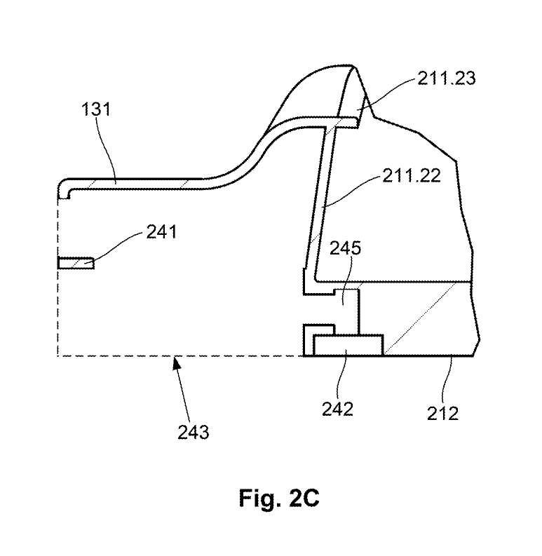

A specific example of a first body will now be described in further detail with reference to FIGS. 2A to 2C.

In this example, the first body 110 includes a generally planar underside 212, with the recess 111 being provided on an upperside of the body 110. The recess is defined by a recess base 211.1 and spaced apart recess side walls 211.2, 211.3 extending generally upwardly from the recess base 211.1. The recess base 211.1 and side walls 211.2, 211.3 are arcuately shaped when viewed from above so as to define outer and inner side walls 211.2, 211.3, which generally conform to a shape of a user's upper jaw. Tabs 211.12 extend rearwardly from the recess base 211.1 to support an insert, and prevent teeth engaging an underside of the insert.

The side walls 211.2, 211.3 are typically smooth and contoured for comfort. The outer side wall 211.2 has a curve profiled outer surface 211.21 which rests against an inner surface of the user's cheeks and is spaced apart from an inner surface 211.22 to define channels (not shown) that extend through the buccal cavity. The recess base 211.1 is further spaced from the underside 212 to define a channel extending between the user's maxillary and mandibular teeth, in use. A front of the outer side wall 211.2 extends forwardly from the body to define an elliptically shaped opening 131, which extends through and beyond the lips of the user, in use, with the opening being in fluid communication with the channels.

The outer side wall 211.2 includes a lip 211.23 extending inwardly from an upper distal edge of the outer side wall 211.2, whilst the inner side wall 211.3 includes a lip 211.33 extending inwardly from an upper distal edge. In use the lips 211.23, 211.33 cooperate to grip inserts provided in the recess, thereby retaining the inserts in position.

The opening 131 includes a cylindrical collar 241 mounted on an inner underside of the opening 131. A socket 245 is provided in the first body 110 within the outer side wall 211.2, in line with the collar at the back of the opening 131, with this extending to a socket opening 242 in the underside 212, allowing an adjustment mechanism to be mounted therein. The lower side of the collar 241 and opening 131 define a carriage slot 243 extending from the collar to the socket opening, allowing a carriage of the adjustment mechanism to move therein. Visual indicia in the form of markings 244 are provided adjacent the carriage slot 243, allowing a position of the second body to be determined, as will be described in more detail below.

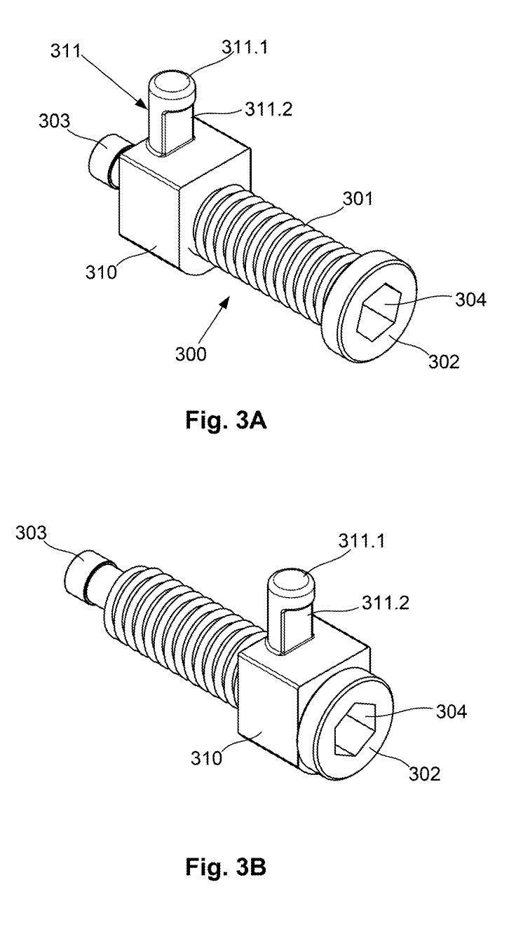

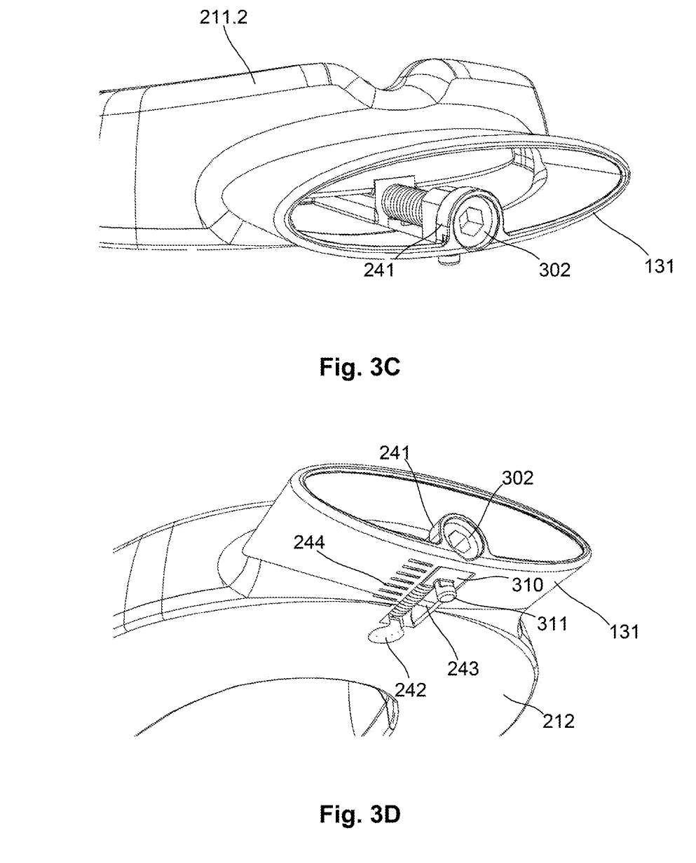

An example of the adjustment mechanism will now be described with reference to FIGS. 3A to 3D.

In this example, the adjustment mechanism 300 includes a threaded shaft 301, having a head 302 provided at one end and a plug 303 provided at the other end. The head 302 can include socket for receiving a key, such as a hex key, allowing the shaft to be rotated in use. A threaded carriage 310 having a generally cubic body is mounted on the threaded shaft allowing this to be moved between the positions shown in FIGS. 3A and 3B as the shaft 301 is rotated. A peg 311 is mounted on the carriage, with the peg including a head 311.1 and shaft 311.2.

In use, the threaded shaft 301 is provided in the first opening 131, with the plug 303 rotatably mounted in the socket 245, and the head 302 positioned within the collar 241, whilst the carriage 310 is located within the carriage slot 243, with the peg 311 projecting outwardly from a lower side of the opening 131. In use, rotation of the head 302 allows the carriage to move longitudinally within the carriage slot 243, with the relative location of the carriage being viewable via the markings 244.

The threaded shaft could be made from any suitable material, and could be printed and/or moulded using plastic, or could be a stainless steel screw manufactured using any suitable technique, whilst the carriage and peg could be formed from plastic using additive manufacturing or the like, depending on the preferred implementation.

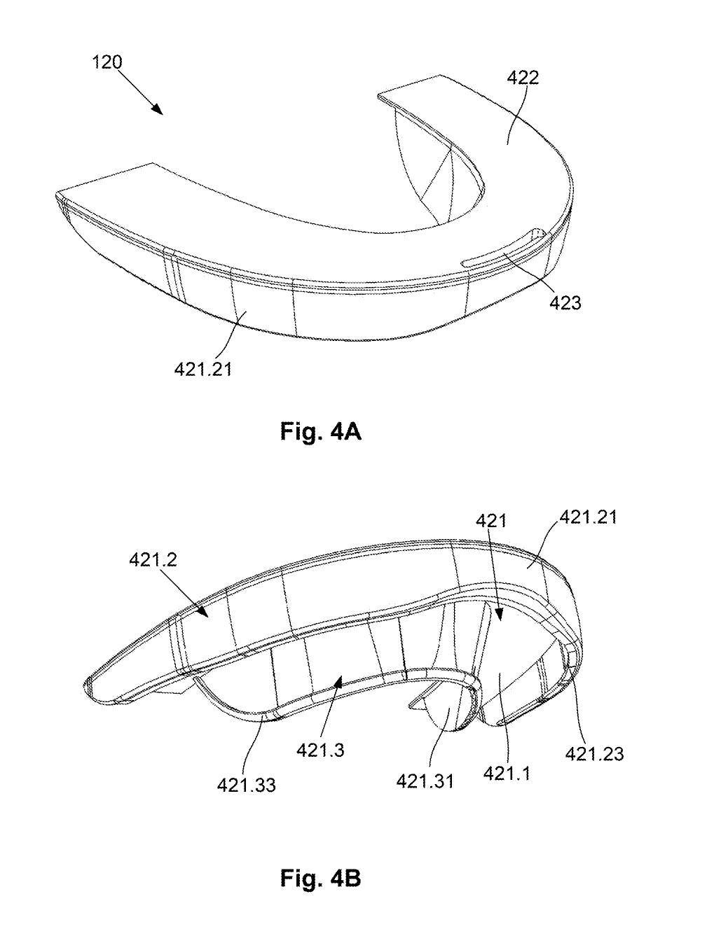

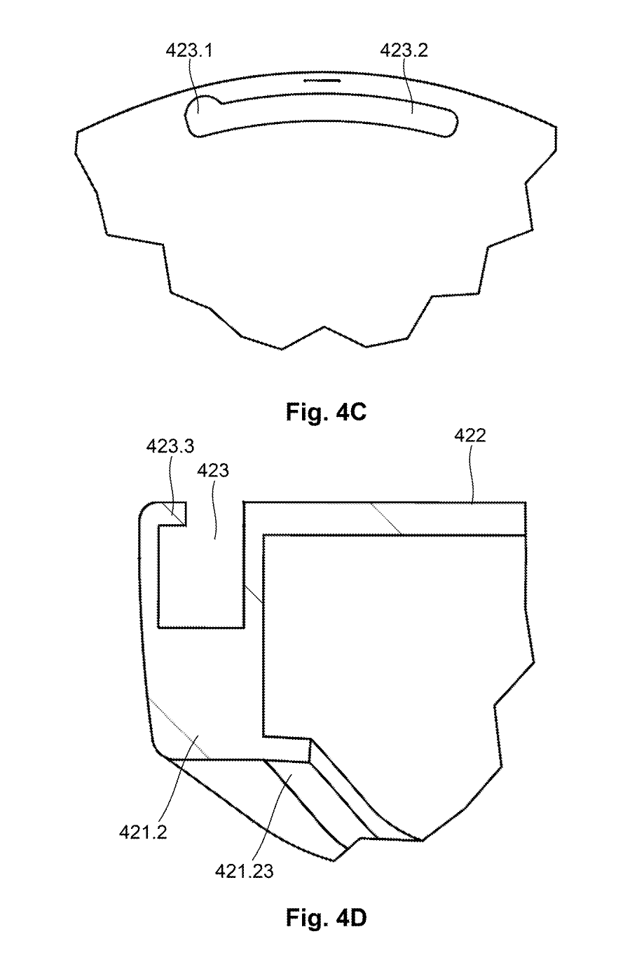

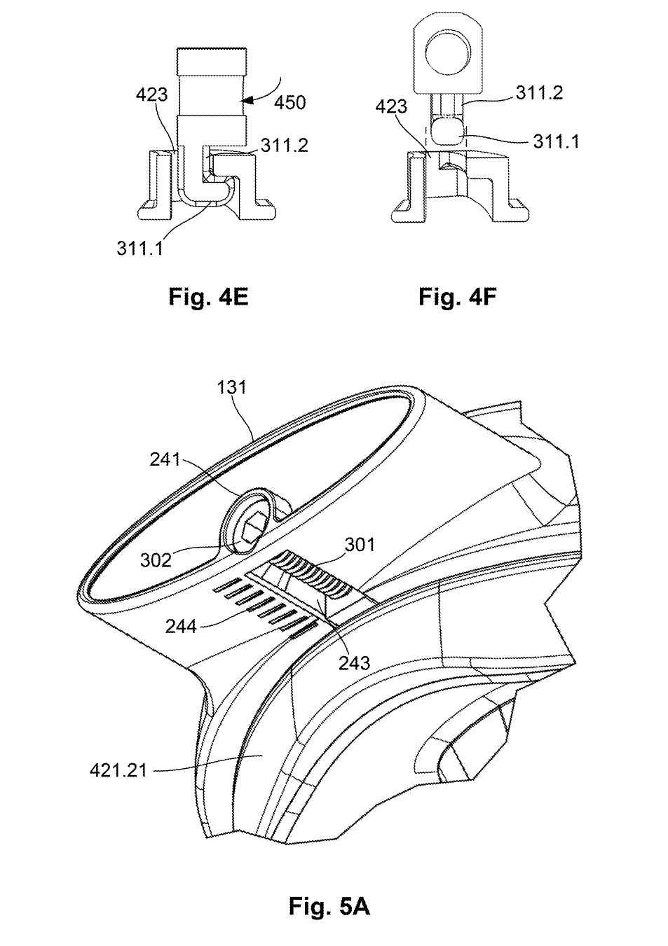

A specific example of a second body will now be described in further detail with reference to FIGS. 4A to 4D.

In this example, the second body 120 includes a generally planar topside 422, with the recess 421 being provided on an underside of the body 110. The recess is defined by a recess base 421.1 and spaced apart recess side walls 421.2, 421.3 extending generally downwardly from the recess base 421.1. The recess base 421.1 and side walls 421.2, 421.3 are arcuately shaped when viewed from below so as to define outer and inner side walls 421.2, 421.3, which generally conform to a shape of a user's upper jaw.

The side walls 421.2, 421.3 are typically smooth and contoured for comfort. The outer side wall 421.2 has a curve profiled outer surface 421.21 which rests against an inner surface of the user's cheeks, whilst the inner side wall 421.3 has a smooth outer surface 421.31 which the user's tongue can rest against in use.

The outer side wall 421.2 includes a lip 421.23 extending inwardly from a distal edge of the outer side wall 421.2, whilst the inner side wall 421.3 includes a lip 421.33 extending inwardly from a distal edge. In use the lips 421.23, 421.33 cooperate to grip inserts provided in the recess, thereby retaining the inserts in position.

The second body 120 also includes a keyhole slot 423 provided near a front of the topside 422, and accommodated within the outer side wall 421.2. The keyhole slot includes an eyelet 423.1 having an eyelet width greater than the head width of the peg head 311.1, allowing the peg to be inserted therein. The slot narrows from the eyelet 423.1 to a narrow end 423.2, defined by a lip 423.3 running along at least part of the slot length. The narrow end is wider than the peg shaft width, but narrower than the head's width, so that the peg head 311.1 abuts against an underside of the lip so that the peg is retained in the slot 423. Additionally the peg shaft length is greater than the height of the lip 423.3, allowing the peg to move up and down within the slot. Accordingly, it will be appreciated that the slot allows for lateral and to and fro movement, whilst ensuring longitudinal movement is constrained.

An alternative arrangement of the slot and peg are shown in FIGS. 4E and 4F. In this example, the peg is configured to sit within the channel in use. However, if rotated through 90°, as shown by the arrow 450, the peg is sufficiently narrow to be removed from the slot. This avoids the need for the keyhole slot design, whilst allowing the peg to be selectively engaged with or removed from the slot through relative 90° rotation of the first and second bodies.

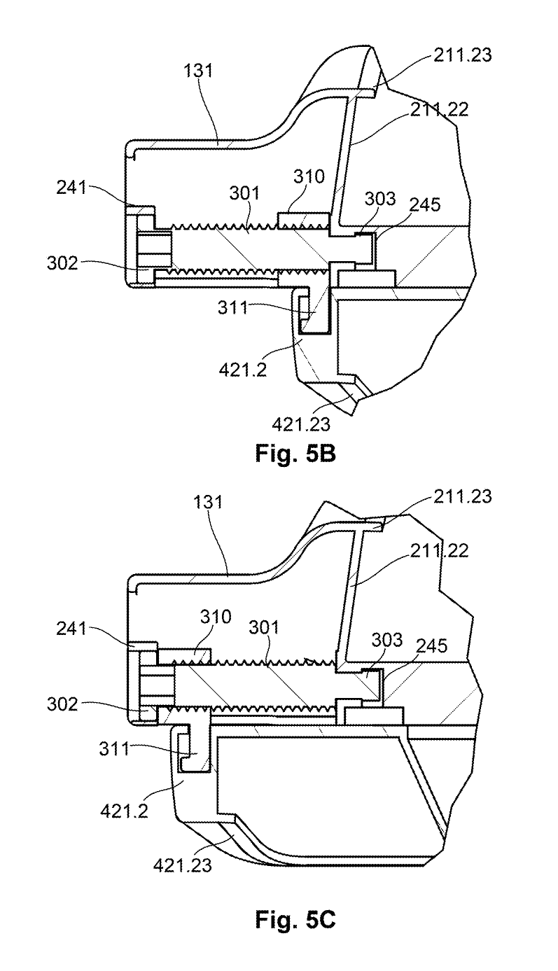

An example of the apparatus with the two bodies connected is shown in FIGS. 5A to 5C, with the second body in the rearward and forward positions in FIGS. 5B and 5C respectively.

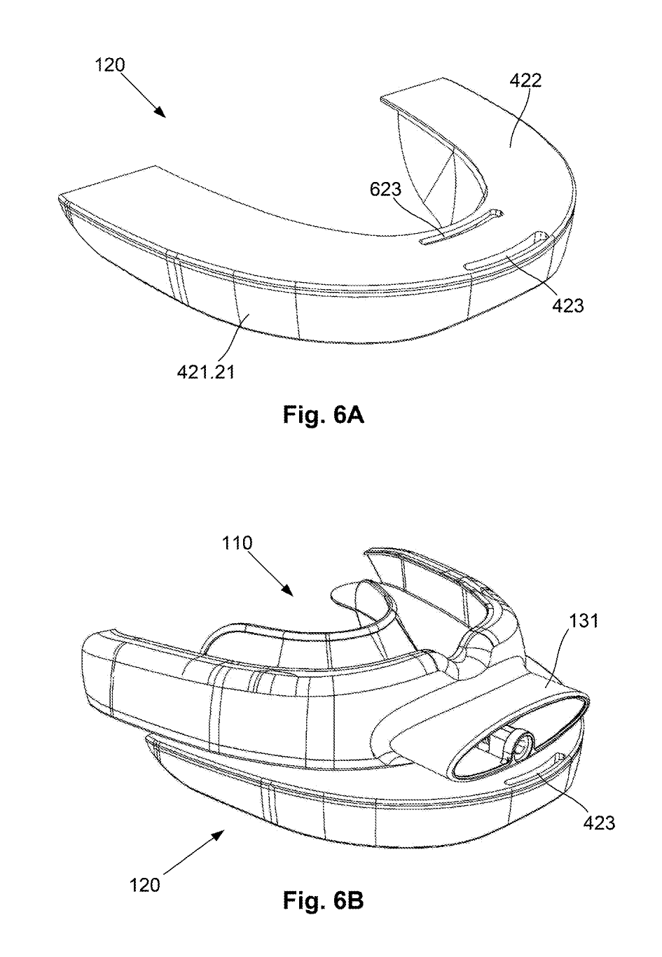

In the above described example, a single slot is provided in the second body. However, this is not essential and multiple slots can be provided, and an example of this will now be described with reference to FIGS. 6A and 6B. For the purpose of this example, similar reference numerals are used to refer to similar features, and these will not therefore be described in detail.

In this example, the second body 120 includes a second slot 623, which is provided spaced rearwardly from the slot 423, allowing the peg to be inserted into either slot. This allows for coarse adjustment of the relative position, through selection of one of the slots, with fine adjustment being provided using the threaded shaft 301. For example, the use of a single slot in one configuration could allow movement of about 7 mm, in which case the use of appropriately spaced dual slots could allow for movement of about 14 mm.

The use of inserts, as described above, allows a variation in the shape of teeth and jaws that can be accommodated by the first and second bodies. This allows the majority of individuals to be fitted by selecting one of a number of defined template bodies, having standard sizes/dimensions. In this regard, as the first and second bodies are separate and only connected using the adjustment mechanism, this allows the first and second bodies to be easily disconnected, with either of the bodies being replaced with a body of a different size. Consequently, providing five standard first body sizes and five standard second body sizes allows twenty five different configurations of apparatus to be provided. In practice, this means the majority of the population can be fitted using a relatively limited number of standard sized bodies, whilst reducing the amount of stock suppliers require to ensure users having different jaw shapes can be accommodated.

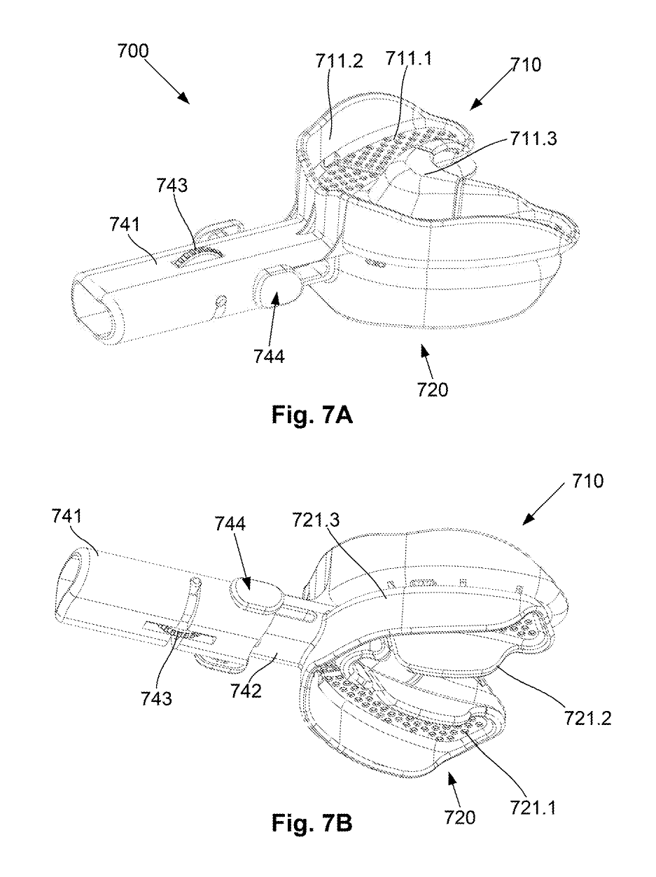

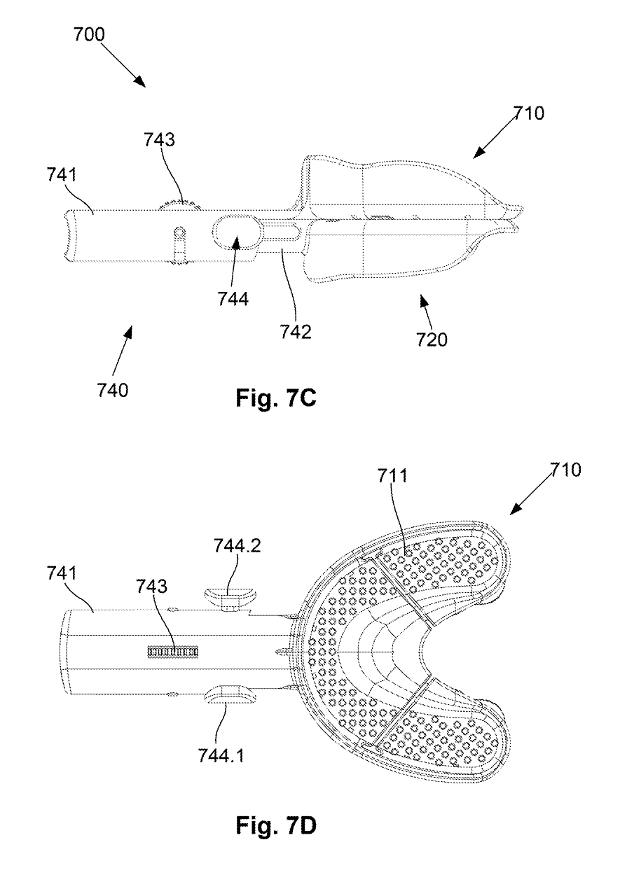

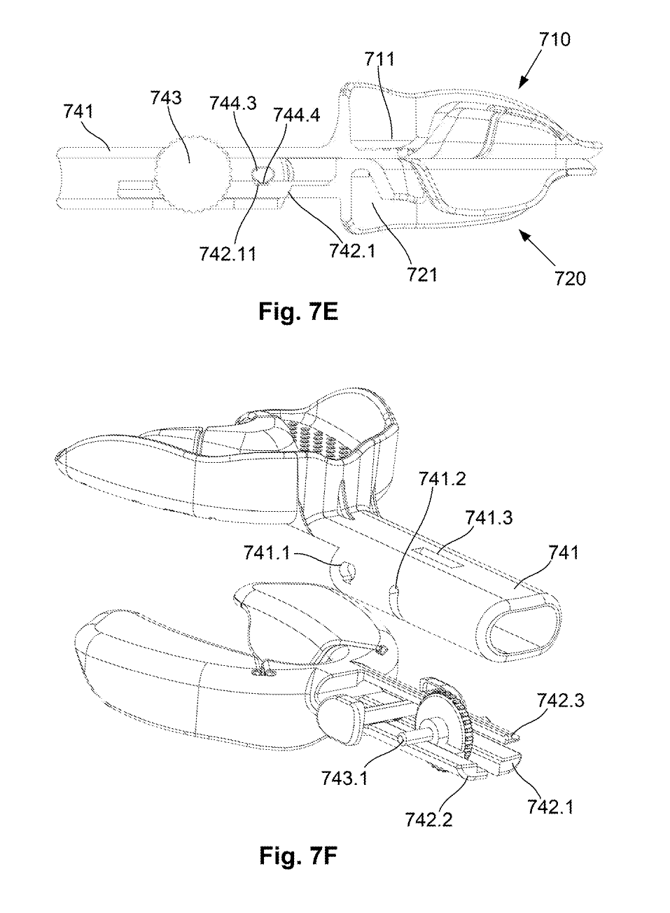

As previously mentioned, the provision of an airway, although advantageous is not essential, and an example of an appliance without an airway will now be described with reference to FIGS. 7A to 7F.

Such appliances can be used to provide assistance in adjusting the relative position of a user's jaws, which can in turn be used for breathing assistance and/or could be used in positioning the jaws while creating inserts, which are then used in an appliance similar to those outlined above.

The current example will also be used to illustrate an alternative adjustment mechanism. However, it should be understood that features of the examples described herein could be used interchangeably, so it will be appreciated that the adjustable mounting of the previous example could be used interchangeably with that of the current example.

In this example, the apparatus 700 includes first and second bodies 710, 720, each including a respective recess 711, 721. In one example, each recess acts as a tray, allowing inserts to be created therein, in which case the recess will be of a form similar to the recesses described above, including a respective recess base 711.1, 721.1, and outer and inner side walls 711.2, 721.2; 711.3, 721.3. The side walls can also include a lip similar to the lips described above, allowing inserts to be created including a shoulder moulded therein, allowing these to engage with recess lips, as previously described, although this is not essential.

In this example, an adjustable mounting 740 is formed from first and second mounting members coupled to the first and second bodies 710, 720, respectively. The first mounting member includes a tubular body 741 defining a sleeve extending forwardly from the first body. The sleeve has a rounded rectangular cross section, although this is not essential and other shapes could be used. The second mounting member 742 includes three arms 742.1, 742.2, 742.3 extending forwardly from the second body 720, with the arms 742.1, 742.3 spaced vertically, whilst the arms 742.1, 742.2 are spaced laterally, with outer edges of the arms being curved so as to abut against internal rounded corners of tubular body 741 when inserted therein, thereby retaining the first and second members 741, 742 in a fixed orientation, whilst allowing relative sliding movement in a longitudinal direction.

A locking member 744 is provided, which in this example includes a shaft 744.3 having button 744.1, 744.2 on either end. The shaft extends laterally through apertures 741.1 in the first mounting member 741, and is supported between the vertically spaced arms 742.1, 742.3, allowing the locking member 744 to shuttle left and right between locked and unlocked positions. The shaft includes a ridge 744.4, which can be received in recesses in the arm 741.1 during insertion, and which otherwise engages against one of the arms 742.1, 742.2, thereby urging the shaft into engagement with the first mounting member 741, so as to frictionally lock the first and second members 741, 742 in position.

A wheel 743 is provided, supported by an axle 743.1, which is rotatably mounted in a wheel mounting aperture 741.2 in the first mounting member 741. The axle engages the first and second arms 742.1, 742.2 so that the wheel rotates upon sliding movement of the first and second mounting members 741, 742, which can be used to provide an indication of a relative position, or to allow sliding movement to be induced through rotation of the wheel. Accordingly, this allows the wheel to act as both an indicator and an actuator.

Accordingly, in one example, inserts can be inserted into the recesses 711, 721, allowing the user's teeth to engage the inserts, with the second body then being moved forward from a rearward position, until a desired amount of mandibular advancement is achieved. Alternatively, curable mould material, such as curable silicone could be provided in the recesses, allowing a user to bite into the material, before the second body is advanced to a desired position. Following this, the apparatus is removed, the moulded material cured and used to manufacture inserts.

In the above examples, the adjustable mounting and in particular the threaded shaft and peg directly engage the first and second bodies 110, 120 respectively. However, the requirement that the threaded shaft 301 rotate within the socket 245, and that the peg is movable within the keyhole slot 423, means the socket 245 and keyhole slot 423 need to be manufactured to a high degree of tolerance, which is not necessarily straightforward using additive manufacturing or moulding.

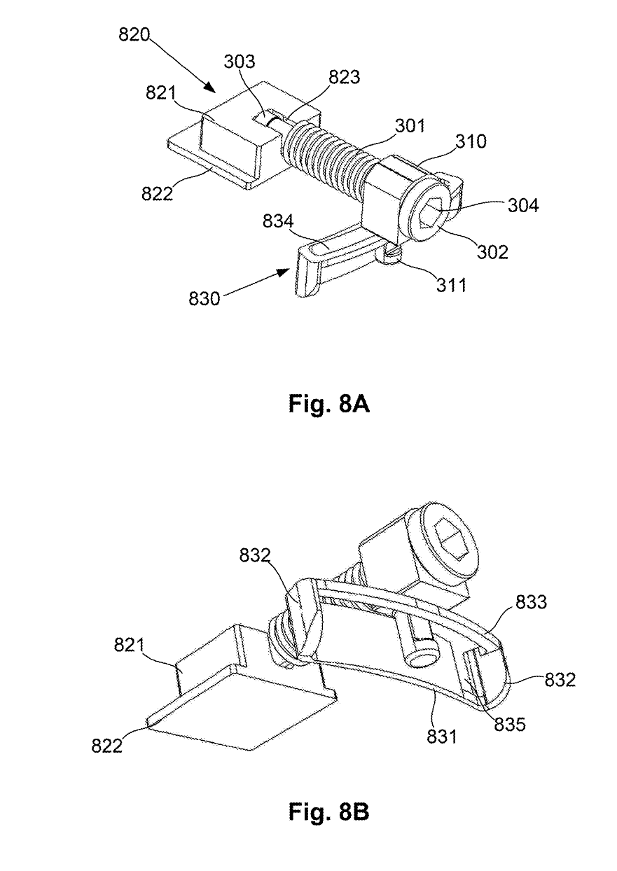

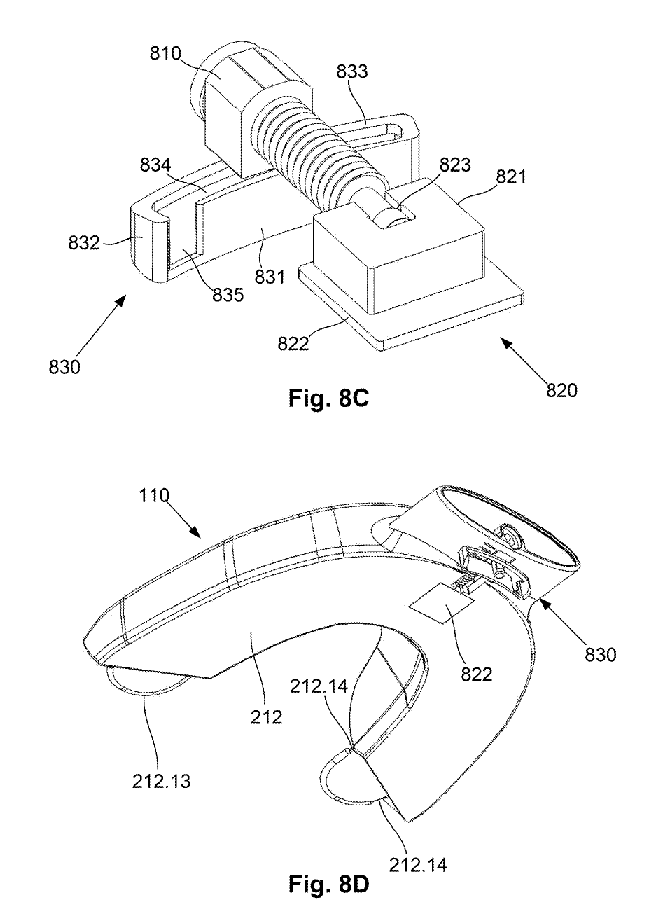

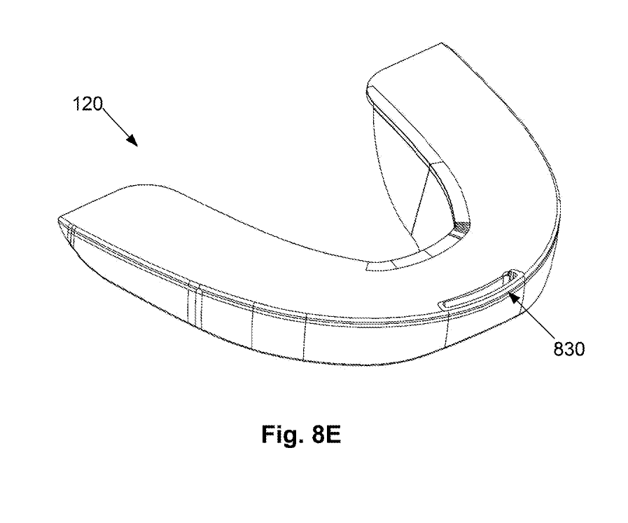

Accordingly, in a further example, the socket and keyhole slot can be made as separate insertable components, which can be provided within the first and second bodies as required. An example of this will now be described with reference to FIGS. 8A to 8E.

In this example, the socket component 820 includes a generally cuboid socket body 821, having a flange 822 extending around at least part of one edge, and incorporating a socket 823. In use, the first body 110 includes a recessed opening, allowing the socket body 821 to be inserted therein, with the flange 822 engaging the recess so that the flange surface is flush with an underside 212 of the first body 110.

The slot component 830 includes an arcuate elongate rear wall 831, having end walls 832 extending forwardly to a front rim 833, spaced from the rear wall 831 so as to define a slot 834. The rear wall includes an opening 835 at one end, thereby defining the eyelet 423.1 allowing the peg 311 to be inserted therein. Again, the slot component 830 is inserted into a suitable opening in the second body 120 to thereby provide the keyhole slot.

It will be appreciated that the above technique allows higher tolerance manufacturing approaches to be used to create the socket and keyhole slot components, with lower more cost effective manufacturing being used for the first and second bodies 110, 120, whilst ensuring smooth and effective operation of the adjustable mounting.

A further feature shown in the example of FIG. 8D, is that the tabs 211.12 that extend rearwardly from the recess base 211.1 to support an insert can include beading 211.13 to avoid sharp edges, but with a gap 211.14 in the beading where the tab joins the recess base, to allow the tab to bend to more closely conform to the shape of the user's jaws.

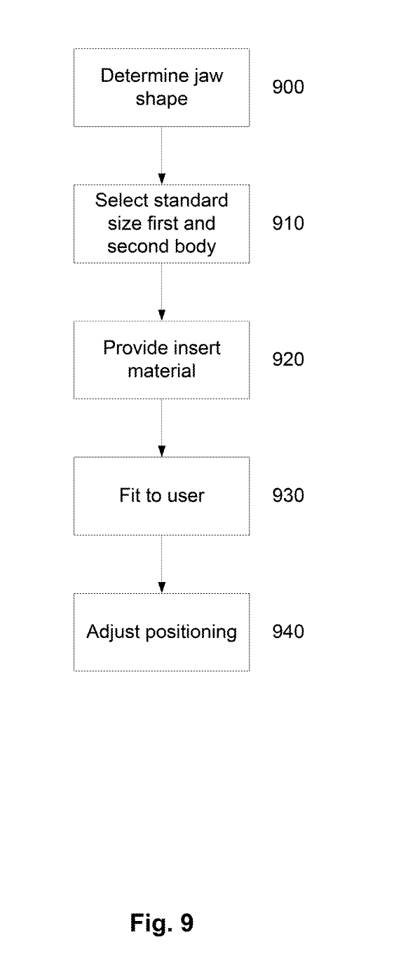

The process of fitting the user with a breathing assist apparatus will now be described, with reference to the fitting process outlined in the flow chart of FIG. 9.

In this example, at step 900 the shape of the user's upper and low jaws are determined. The shape information can include dimensions of the jaws of the user, including width and/or curvature information. The shape can be derived from a scan, such as a CT scan of the user's oral cavity, or alternatively can be obtained from dental impressions, 3D models, 3D scans of the user's teeth, cone beam imaging, moulds, digital impressions of an intra-oral scan, or the like. Alternatively, the dimensions could be obtained from a series of photos of the patient's mouth or impressions taken with a smart phone or the like. In a further example, this can be achieved by having the user bite into a marked sheet of material, such as paper or the like, or by measuring the user's jaws with callipers or other suitable measurement apparatus.

At step 910, standard sizes of upper and lower bodies are selected, with insert material being provided into the recesses at step 920. The insert material is typically a curable material, such as a heat, chemically or optically cured material, including but not limited to silicone, EVA, polymer resin or the like. The apparatus is placed in the user's oral cavity, allowing the user to bite into the insert material, moulding this to the shape of the user's jaws and teeth at step 930. The apparatus is then removed from the user's mouth and the insert material cured and optionally finished to remove edges and extraneous material.

Following this, the apparatus is returned to the user's mouth and the position of the first and second bodies adjusted as required at step 940. In particular, the second body will typically be moved progressively forward from the most rearward position, until the airway of the user is cleared or the apparatus begins to cause discomfort.

Thus, it will be appreciated that the above described arrangement allows an apparatus to be rapidly fitted from a more limited range of different standard sized bodies compared to if a single unitary body appliance were used.

Furthermore, when performing the fitting, the practitioner can arrange for two sets of inserts to be made using the silicone or other curable material. One set of inserts can then be used as a temporary set of inserts, whilst the other is subsequently scanned and used to create more permanent inserts, for example by using additive manufacturing to create acrylic based inserts, which are typically more durable. Thus, the ability to use a curable material allows inserts to be created on the spot, so a user can be provided with an appliance during an initial consultation, with semi-permanent inserts being subsequently supplied to provide a more resilient and longer lasting configuration.

Additionally, semi-permanent inserts can be manufactured so as to be thermoformable, allowing these to be reshaped slightly by heating to accommodate changes in the user's jaw positioning or shape over time.

Even such semi-permanent inserts would typically undergo wear and potential discolouration, and hence can be replaced periodically. Despite this, the first and second bodies can be reused as needed, so inserts can be recreated from the previously scanned moulds. Furthermore, as the first and second bodies are standard sized and can be formed from a durable material such as titanium, these can even be reused by other individuals in the event that a first user no longer requires the apparatus.

The ability to remove inserts allows these to be replaced and/or cleaned and re-used as required. Similarly, the bodies can also be cleaned and/or sterilised prior to re-use.

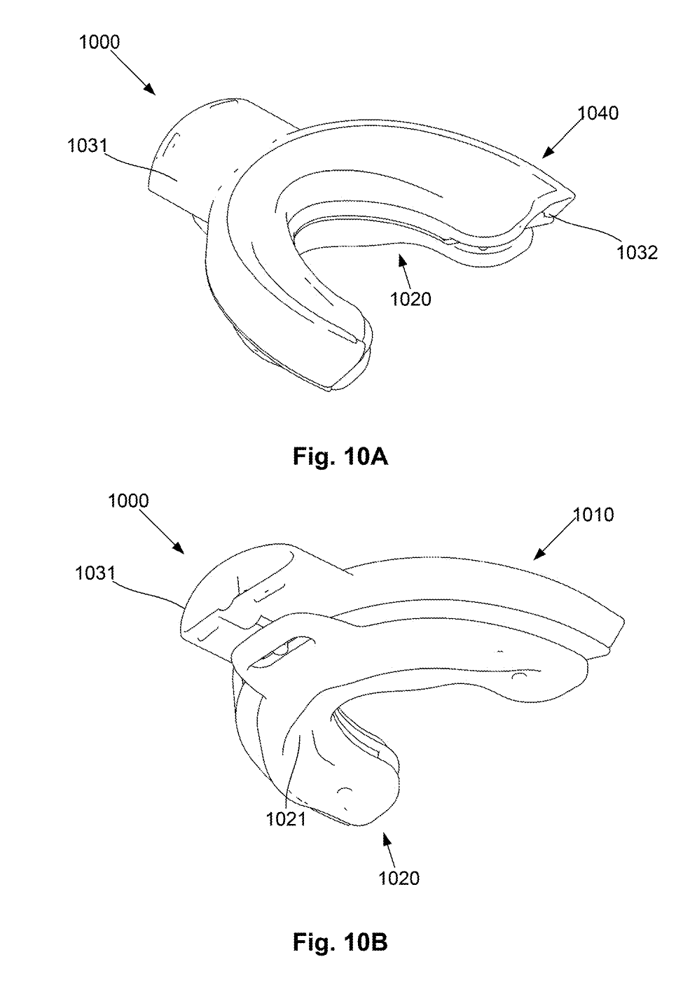

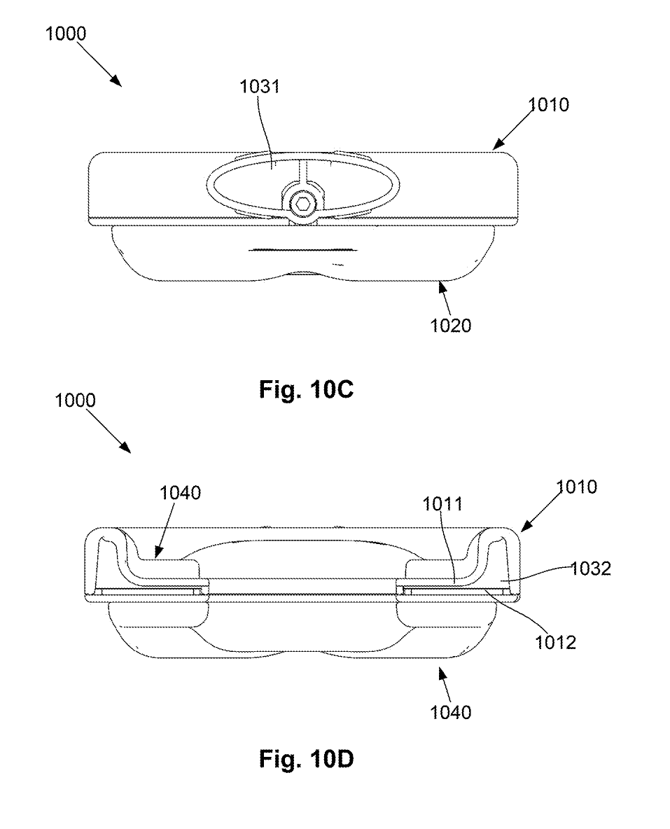

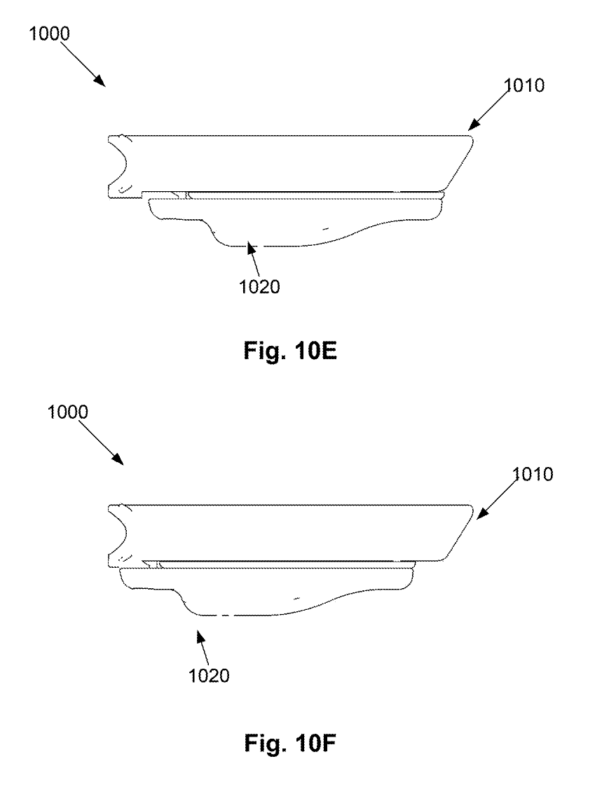

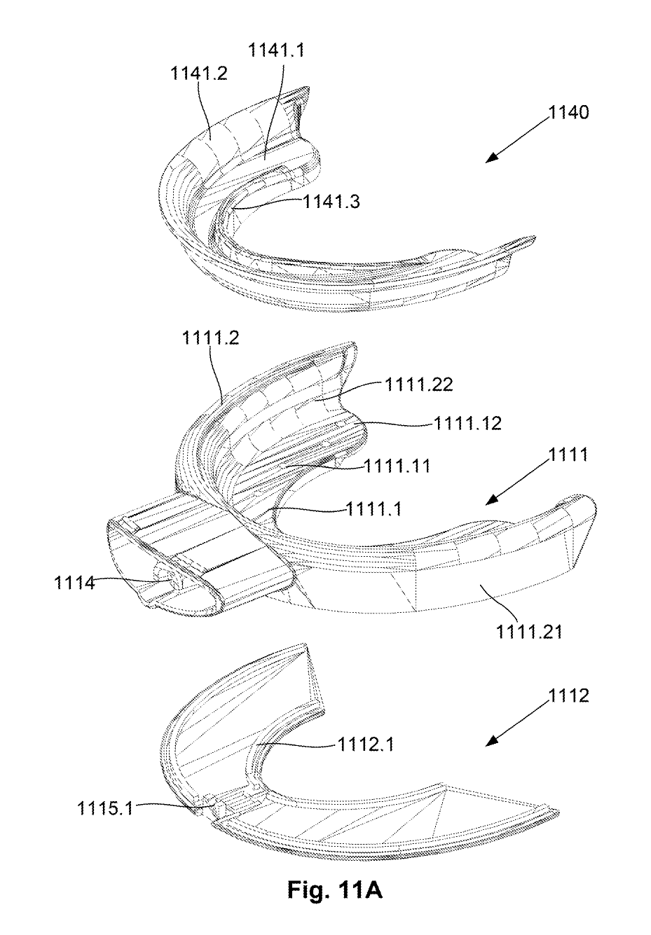

In a further example, the bodies can be manufactured using injection moulding. An example of an apparatus for providing breathing assistance will now be described with reference to FIGS. 10A to 10F.

In this example, the apparatus 1000 includes a first body 1010 and a second body (not shown). The first body includes a base 1012 and a cover 1011, which cooperate to define at least one first opening 1031 for allowing airflow between the lips of the user, and at least one second opening 1032 provided in the oral cavity of the user, the first and second openings being interconnected via one or more channels (not shown) to allow airflow into and out of a posterior region of the oral cavity.

Each of the first and second bodies is coupled to a respective insert 1040, which is moulded to fit the user's teeth, as will be described in more detail below. The term insert will be understood to include any form of member that is shaped to conform to, or otherwise suitably receive, the user's teeth. In some instances the insert is referred to as an “insert”, and the terms should be considered as being interchangeable. This arrangement allows the apparatus to be positioned in and retained within the user's mouth in use.

Thus, the device has a first opening 1031, which can extend beyond the lips, or at least keep the lips apart, to allow airflow therethrough. Air passes through airways defined by the channels, and is directed into a posterior region of the mouth through second openings 1032, bypassing issues with tongue and lower jaw position. In more severe cases, the first opening 1031 can be connected to an external device, such as a CPAP (Continuous Positive Airway Pressure) machine, air supply, or the like providing more comfort and increased patient compliance compared to a mask. In such situations, the airway 1031 and 1032 may be divided into two or more airwaves working together or in opposite directions.

Providing air flow directly into a posterior portion of the user's oral cavity has a number of benefits. In particular, this avoids obstructions created by the nasal cavity, soft palate and tongue which can lead to snoring and apnoea events, and helps reduce the drying effects of air flow, which can in turn lead to user discomfort. This makes the apparatus comfortable to wear whilst ensuring an unobstructed air flow thereby preventing snoring and apnoea events. Thus, for example, nasal obstructions can be bypassed by air flow through the apparatus, thereby bypassing the nasal airway or adding to it in the case of a partial obstruction. Furthermore, air flowing below or on both sides of the soft palette helps prevent collapse of the soft palate, which can in turn lead to additional obstruction.

A number of further features will now be described.

The base 1011 can be a substantially planar base, optionally including ridges or guides to facilitate attachment to the cover, whilst the cover is shaped to define the channels, the openings and to allow attachment of the insert. The base and cover can be connected via any appropriate mechanism, such as a mechanical coupling, a friction fit, interference fit, adhesive, ultrasonic welding or the like.

In one example, the bodies are manufactured using injection moulding, and in particular by injection moulding a polymer, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials. The formation of the first body from the base and cap facilitates the process of injection moulding the body ensuring that the channels can be formed, which would be an otherwise difficult process.

The bodies can be coated with a medical grade polymer and in one example, a medical grade elastomer, such as silicone or polyurethane, epoxy or parylene, for improved comfort as well as ensuring biocompatability.

Typically one or more standard sizes of first and second body can be produced, with an appropriate first and second body being selected based on a closest fit to the intended user. Custom fitting can then be achieved using customised inserts positioned between the user's teeth and the respective body in use. Each insert is typically customised for a user's teeth and is adapted to be removable and/or replaceable. Additionally, the first and second bodies can be resized, for example by cutting or otherwise removing part of the body, and in particular tabs provided on a rearward part of the first body, as will be described in more detail below.

Inserts can also be made by injection moulding similar materials to the bodies, could be made by additive manufacturing, such as 3D printing, and/or could be manufactured by having the user bite into a material that can be moulded to the shape of the user's teeth and then solidified. For example, this could include UV curing, using a thermosetting material or the like. In one example, the inserts are formed from boil and bite materials, such as Ethylene-vinyl acetate or the like, although silicone or other materials, such as a thermosetting polymer, a thermoplastic polymer, silicone, an elastomer, polyvinylsiloxane, polyurethane, ethylvinylacetate, polycarbonate, acrylonitrile butadiene styrene, or a combination of these materials, could be used. For example, this could be used to allow a user to mould the inserts at home by biting into a bite into a member made of a suitable material such as silicone.

In one example, the apparatus can be used be with a plurality of different inserts, which can be used for example to provide different levels of fit, comfort, support or the like. The inserts can also be either temporary or semi-permanent, and may be made from different materials depending on their intended use. For example, a temporary insert could be created upon the initial fitting of a breathing assist apparatus, using a silicone which is moulded in-situ, with this being replaced by a subsequent semi-permanent insert, such as an injection moulded insert, once there has been opportunity for this to be manufactured. This allows an initial fitting to be performed when the apparatus is initially supplied with temporary inserts, with semi-permanent acrylic inserts being subsequently manufactured and provided to the user once ready.

Whilst the inserts could be fitted using any suitable technique, in one example, the inserts can be attached to the first and second bodies using adhesives, mechanical couplings, such interference fits, or the like.

The use of inserts, as described above, allows a variation in the shape of teeth and jaws that can be accommodated by the first and second bodies. This allows the majority of individuals to be fitted by selecting one of a number of defined template bodies, having standard sizes/dimensions.

Additionally, inserts can be manufactured so as to be thermoformable, allowing these to be reshaped slightly by heating to accommodate changes in the user's jaw positioning or shape over time. Even such semi-permanent inserts would typically undergo wear and potential discolouration, and hence can be replaced periodically. Despite this, the first and second bodies can be reused as needed, so inserts can be recreated from the previously scanned moulds. The ability to remove inserts allows these to replace and/or cleaned and re-used as required. Similarly, the bodies can also be cleaned and/or sterilised prior to re-use.

When an airway is provided, the channels can have a wide variety of configurations and may be sized and shaped depending on the anatomy of the oral cavity of the user. This is typically done to maximise the available airway, whilst ensuring comfort for the user. In one example, the total airway has a cross sectional area of at least 50 mm², at least 70 mm², at least 90 mm², at least 100 mm² and at least 110 mm². The dimensions selected will vary depending on a wide range of factors, including whether the device is required to provide a partial or complete airway, for example to bypass a partial or complete blockage. Additionally, this will depend on the intended use and the associated airflow requirements. Typically the dimensions of the channels and/or openings 1031, 1032 are selected so that in conjunction with the user's existing airways, the total airway available corresponds to the cross sectional area of an airway in a healthy subject for both nasal and pharyngeal airways. In any event, the cross sectional areas used will depend on the preferred implementation and intended use, so for example, a smaller cross sectional area may be used for children, adolescents, or individuals with only partial obstructions. In contrast increased cross sectional areas may be used where a high flow rate is required, for example in the event that the device is to be used to provide breathing assistance during exercise.

In one example, each channel includes a portion extending through the buccal cavity, between the user's cheeks and teeth, and a portion extending between the user's maxillary and mandibular teeth. This arrangement maximises the cross sectional area of the channels, whilst maintaining comfort for the user, by distributing the airway between the user's teeth and cheeks, and between the user's teeth.

The cross sectional area of the first and second channel portions can vary between the first and second openings, allowing the overall cross section of the channel to be maintained, whilst having the channel conform as far as possible to the natural space available in the oral cavity. It will be appreciated that any variation can be used, depending for example on the configuration of the user's oral cavity.

In one example, the second openings are angled inwardly at between 10° and 50°, more typically between 20° and 40° and preferably about 30° to assist in airflow into and out of the oral cavity, and in particular to direct airflow towards the centre of the oral cavity. Additionally and/or alternatively, the second openings are positioned over the last or back tooth on each side of the top jaw. As previously mentioned, the provision of an airway, although advantageous is not essential.

In one example, the apparatus further includes an adjustable mounting configured to interconnect the first and second bodies to allow a relative position of the first and second bodies to be adjusted. The form of the adjustable mounting will vary depending upon the preferred implementation and specific examples will be described in more detail below.

Thus, the apparatus provides an oral appliance for providing breathing assistance. This can be used during sleep, for example for the treatment of both, snoring and sleep apnoea, and can also be used at other times, for example in the treatment of respiratory conditions, such as emphysema, and to assist in jaw placement to provide an adequate airway, for use during surgery, CPR (Cardiopulmonary resuscitation), or the like.

As respective sets of teeth of the user are provided in the inserts coupled to the first and second bodies, adjusting the relative position of the first and second bodies can be used to selectively position the mandibular teeth relative to the user's maxillary teeth, for example to provide mandibular advancement. In this regard, it is known that mandibular advancement can assist in holding open the user's airway, which in turn can reduce snoring. For example, temporomandibular joint disorder (TMD) arises when the upper and lower jaws are misaligned. This may be naturally occurring or can result from injury, or the like. Regardless, such jaw misalignment tends to contribute to airway obstructions by changing the shape of the upper airway, and moving the tongue towards the posterior of the oral cavity, which can in turn exacerbate issues associated with OSA and snoring. Accordingly, by allowing the relative position of the first and second bodies to be adjusted, this allows the jaws of the user to be aligned thereby reducing the effects of TMD, and hence further reducing the likelihood of snoring and OSA.

Accordingly, the provision of the adjustable mounting, allowing the relative position of the first and second bodies to be controllably adjusted, in turn allows a relative position of the lower jaw to be adjusted, for example to provide for mandibular advancement. This can assist significantly in the reduction of snoring.

In one example, the apparatus allows a relative position of the first and second bodies to be adjusted in a longitudinal direction, with the second body being positionable at points between rearward and forward positions shown in FIGS. 10E and 10F respectively. In this regard the longitudinal direction is typically aligned with a dental midline of the user in use, so that this configuration can provide mandibular advancement, although it will be appreciated that other forms of adjustment could be provided.

The adjustable mounting is typically configured to further allow relative lateral movement of the first and second bodies, and to allow relative to-and-fro movement of the first and second bodies. These movements can be largely unconstrained, allowing a user to move their jaw laterally, or to open and close their jaws, whilst remaining engaged with the apparatus. This can aid comfort when using the device, whilst ensuring mandibular advancement is maintained.

The adjustable mounting can be of any appropriate form. In one example, this includes a threaded shaft rotatably mounted to the first body, arranged to extend in a longitudinal direction and which has a threaded carriage mounted thereon to allow the carriage to move longitudinally in response to rotation of the shaft. The carriage supports a peg, which in use engages a slot in the second body, allowing the second body to move longitudinally as the shaft is rotated.

In one example, the slot is a keyhole slot, having a wide end defining an eyelet into which the peg can be inserted, and a narrow section for retaining the peg in place. The slot can have a slot depth greater than a head height of the peg, and wherein the lip has a lip height smaller than a shaft height of the shaft to allow the peg to move in the slot to allow for relative to and fro movement of the first and second bodies.

In one example, the threaded shaft is mounted at least partially within the first opening in the first body. To achieve this, the threaded shaft can include a plug that is rotatably mounted within a corresponding socket in the first body, whilst the threaded shaft includes a head at a second end, which allows the shaft to be rotated, for example through engagement with a tool such as a hex key or similar. The first body typically also includes a collar extending at least partially around the threaded shaft approximate the second end with the head being recessed within the collar, to thereby hold the second end in position, whilst allowing rotation of the head end.

Accordingly, it will be appreciated that the combination of the airway and the ability to adjust the relative position of the first and second bodies can provide assistance beyond that afforded by either the airway or mandibular advancement alone. However, it will also be appreciated from examples described in more detail below that the airway is not necessarily required depending on the intended usage of the arrangement. In this regard, a similar configuration of adjustable bodies can be used for the purpose of adjusting a relative position of the use's jaws, for example to correct muscular-skeletal defects and/or for use in creating moulded inserts for use with an oral appliance, as will be described in more detail below.

It will be appreciated that the overall configuration of the apparatus described above, when the first and second bodies are combined, is similar to arrangements described in copending applications PCT/AU2012/000565 and PCT/AU2015/050144, the contents of which are incorporated herein by reference.

For example, the apparatus can include a filter for filtering air flowing through the apparatus. This can help remove particulates, pollen or other contaminants entrained in air flowing into the device, which can assist in reducing respiratory irritation, which can in turn exacerbate snoring and breathing difficulties. The filter can be positioned anywhere within the body 110, but is typically provided within the first opening 131, thereby allowing this to be easily removed and replaced if required. The filter could be of any suitable form and could include a porous plastic or cloth based filter, and may include additional materials for added functionality. For example, the filter can also include activated carbon for filtering out pollution/bacteria.

Additionally, and/or alternatively a heat/moisture exchanger can be provided that controls the water and temperature content of the air being inhaled by exchanging heat and moisture with exhaled air. Examples of such exchangers can be found for example in U.S. Pat. No. 5,433,192, and these will not therefore be described in any further detail.

Additionally or alternatively, the apparatus can include a valve (not shown) for regulating air flow through the apparatus. In one example, this can be used to resist outflow of air from the second openings to the first opening. This can assist in regulating breathing and in particular allow for rapid inhalation, whilst ensuring slower exhalation, thereby optimising gas exchange within the lungs, for example to minimise the chances of hyperventilation. The valve can be of any suitable form, such as a ball valve, umbrella valve, or the like, and can be adjustable or titratable to ensure that the level of flow control is appropriate to the user.

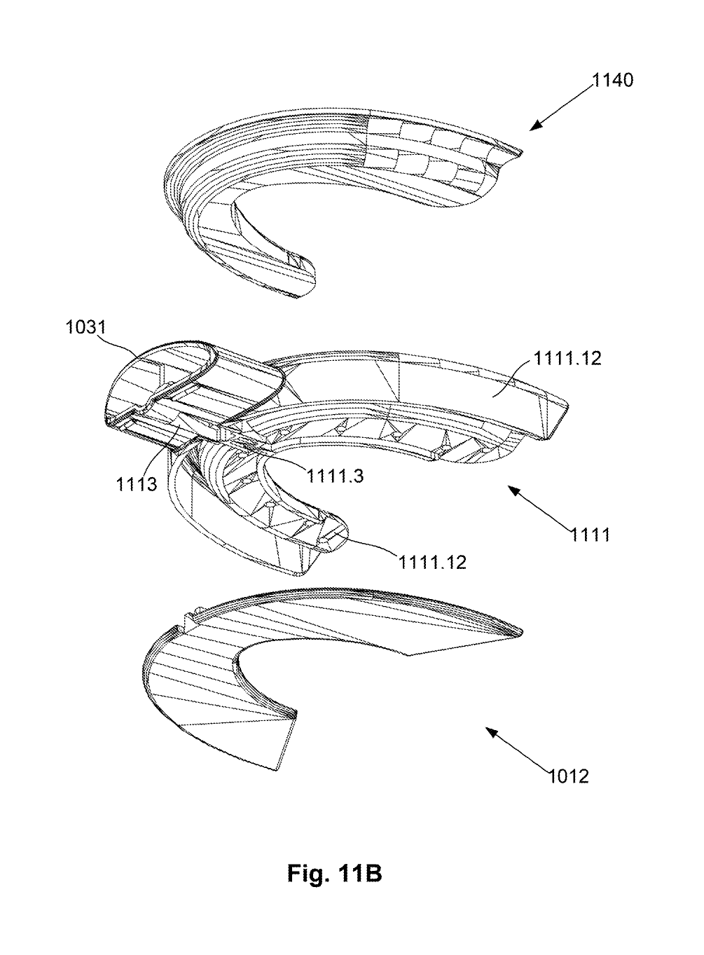

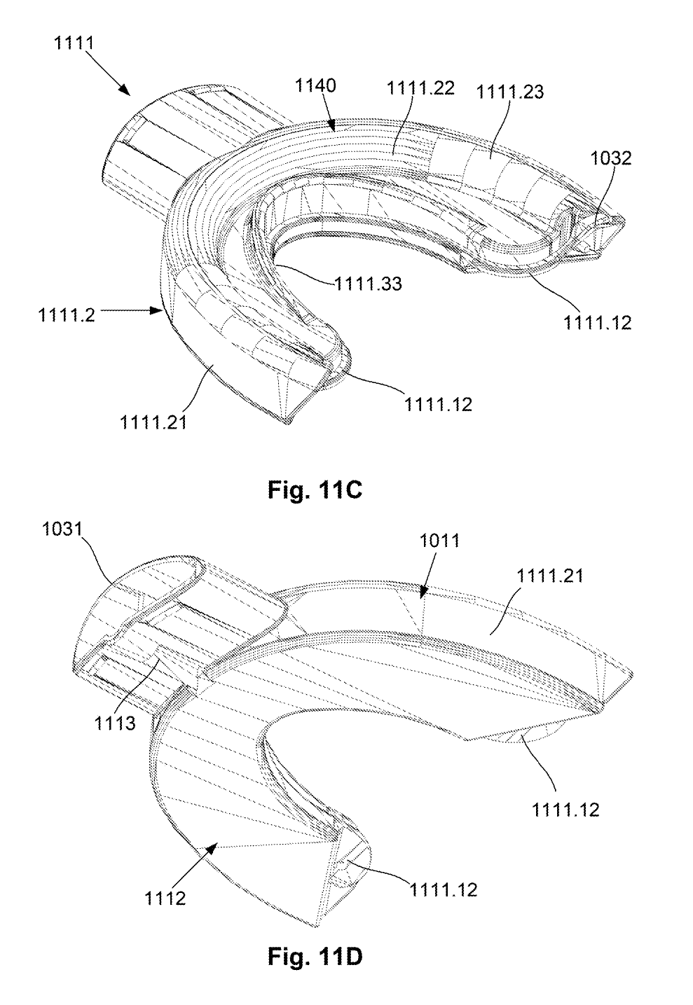

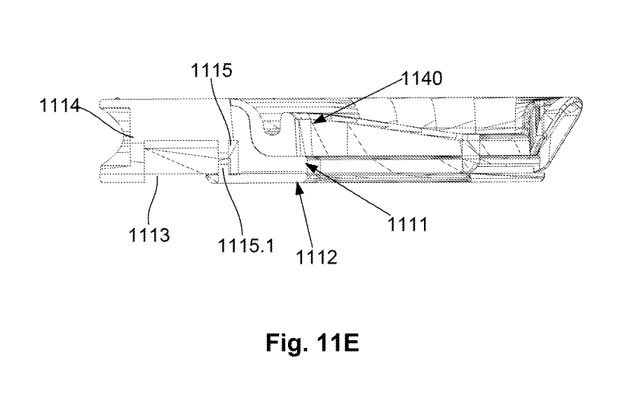

A specific example of the first body and first insert will now be described in further detail with reference to FIGS. 11A to 11E.

In this example, the first body 1010 includes a base 1112 defining a generally planar underside, and a cover 1111 profiled to define the channel. The base 1112 typically includes ridges 1112.1 that facilitate positioning of and attachment to the cover 1111, for example through adhesion, ultrasonic welding or the like. The cover typically includes an insert surface 1111.1 and sidewalls 1111.2 extending generally upwardly from the insert surface 1111.1. The insert surface 1111.1 and side wall 1111.2 are arcuately shaped when viewed from above so as to conform to a shape of a user's upper jaw. Tabs 1111.12 extend rearwardly from the insert surface 1111.1 to support the insert, and prevent teeth engaging an underside of the insert.

The cover 1111 also typically includes a block or support 1111.3 positioned rearwardly of the first opening 1031, which engages the base 1112. This provides additional support, and can assist in preventing crushing of the cover 1111, and hence channel 1033, when a user bites down on the apparatus, for example during in-situ moulding of the inserts. It will be appreciated that additionally or alternatively, the support could be provided on the base.

The side wall 1111.2 is typically smooth and contoured for comfort. The outer side wall 1111.2 has a curve profiled outer surface 1111.111 which rests against an inner surface of the user's cheeks and is spaced apart from an inner surface 1111.22 to define channels (not shown) that extend through the buccal cavity. The insert surface 1111.1 is further spaced from the base 1112 to define a channel extending between the user's maxillary and mandibular teeth, in use. A front of the outer side wall 1111.2 extends forwardly from the body to define an elliptically shaped opening 1031, which extends through and beyond the lips of the user, in use, with the opening being in fluid communication with the channels.

The cover 1111, and in particular the insert surface 1111.1 can include holes 1111.11 or other features to facilitate engagement of the respective insert 1140. In particular these can be used to allow fasteners to be attached to the insert, or to allow the insert to be provided within the holes, for example if the insert is formed in situ, thereby forming mechanical couplings. Additionally or alternatively, the insert can be attached using adhesives, or over-moulding of the insert.

The insert 1140 further defines a recess that receives the user's teeth including a bite base 1141.1 and spaced apart bite sidewalls 1141.2, 1141.3 extending generally upwardly from the bite base 1141.1. The bite base 1141.1 and bite side walls 1141.2, 1141.3 are arcuately shaped when viewed from below so as to define outer and inner side walls 1141.2, 1141.3, which generally conform to a shape of a user's upper jaw. The side walls 1141.2, 1141.3 are typically smooth and contoured for comfort.

The opening 1031 includes a cylindrical collar 1114 mounted on an inner underside of the opening 1031. A socket 1115 is provided in the first body 1010 within the outer side wall 1111.2, in line with the collar at the back of the opening 1031, with this being defined in part by a socket portion 1115.1 extending upwardly from the base 1112, allowing an adjustment mechanism to be mounted therein when the base 1112 is coupled to the cover 1111. The lower side of the collar 1114 and opening 1031 define a carriage slot 1113 extending from the collar to the socket opening, allowing a carriage of the adjustment mechanism to move therein. Visual indicia in the form of markings can be provided adjacent the carriage slot, allowing a position of the second body to be determined, as will be described in more detail below.

The adjustment mechanism used is similar to that previously described with respect to FIGS. 3A to 3D and this will not therefore be described in further detail.

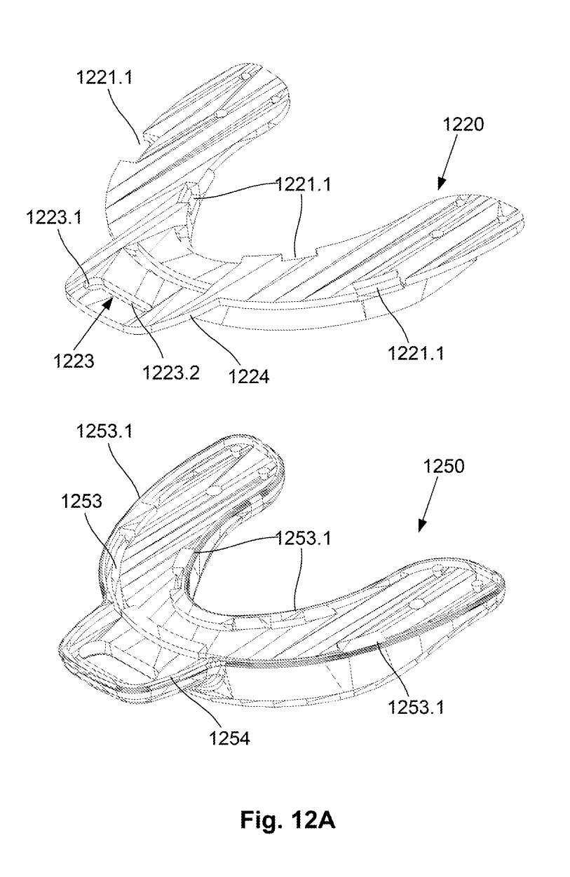

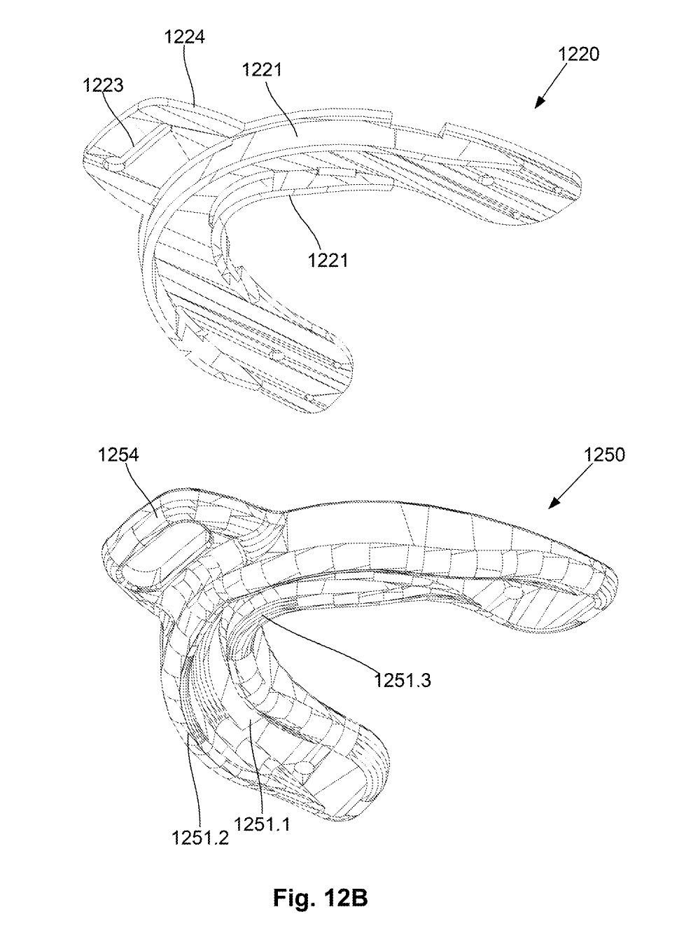

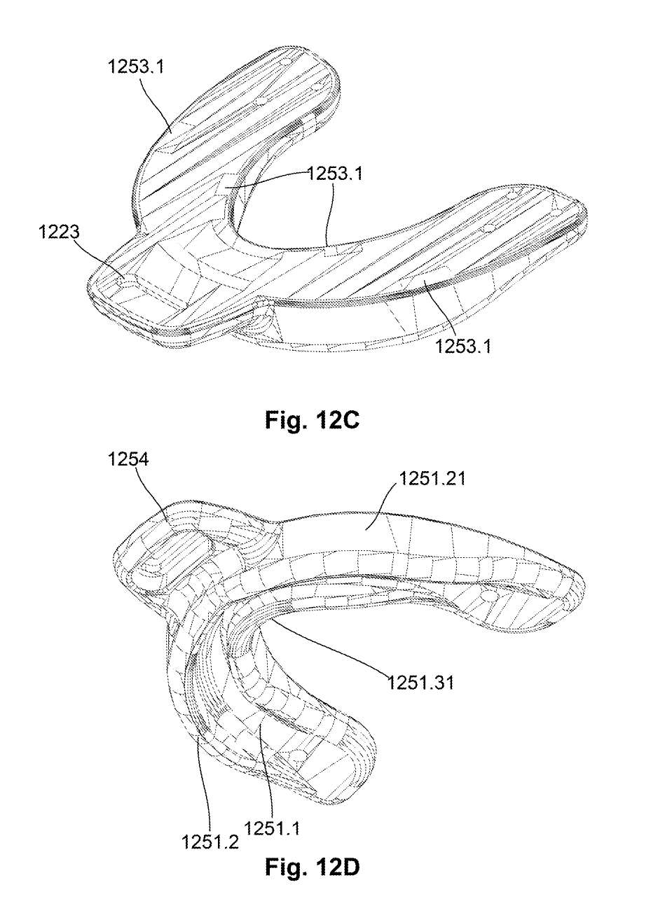

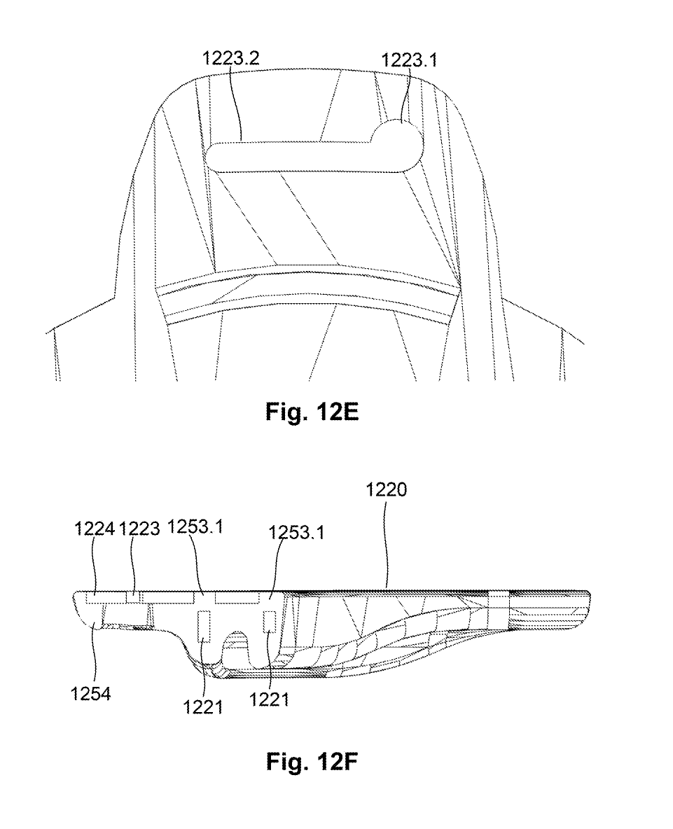

A specific example of a second body will now be described in further detail with reference to FIGS. 12A and 12F.

In this example, the second body 1220 includes a generally planar member including surface ridges 1221 extending downwardly from opposing edges of an underside, with cut-outs 1221.1 being provided behind the ridges on an upperside of the second body 1220 that cooperate with the respective insert 1250, to facilitate positioning and interconnection of the second body and the insert. In particular, the insert 1250 includes overmoulded tabs 1253.1 and channels 1253, with the channels 1253 receiving the ridges 1221, whilst the tabs 1253.1 engage the cut-outs 1221.1 to couple the insert to the second body 1220. A cover tab 1254 extends from a front of the insert 1250 to cover the flange 1224, with the cover tab including an aperture 1254.1 that aligns with the slot 1223.

The insert 1250 further defines a recess that receives the user's teeth including a bite base 1251.1 and spaced apart bite sidewalls 1251.2, 1251.3 extending generally downwardly from the bite base 1251.1. The bite base 1251.1 and bite side walls 1251.2, 1251.3 are arcuately shaped when viewed from below so as to define outer and inner side walls 1251.2, 1251.3, which generally conform to a shape of a user's lower jaw. The side walls 1251.2, 1251.3 are typically smooth and contoured for comfort. The outer side wall 1251.2 has a curve profiled outer surface 1251.21 which rests against an inner surface of the user's cheeks, whilst the inner side wall 1251.3 has a smooth outer surface 1251.31 which the user's tongue can rest against in use.

The second body 1220 also includes a keyhole slot 1223 in a flange 1224 projecting from a front of the body. The keyhole slot includes an eyelet 1223.1 having an eyelet width greater than the head width of the peg head 311.1, allowing the peg to be inserted therein. The slot narrows from the eyelet 1223.1 to a narrow end 1223.2, defined by a lip 1223.3 running along at least part of the slot length. The narrow end is wider than the peg shaft width, but narrower than the heads width, so that the peg head 311.1 abuts against an underside of the lip so that the peg is retained in the slot 1223. Additionally the peg shaft length is greater than the height of the lip 1223.3, allowing the peg to move up and down within the slot. Accordingly, it will be appreciated that the slot allows for lateral and to and fro movement, whilst ensuring longitudinal movement is constrained.

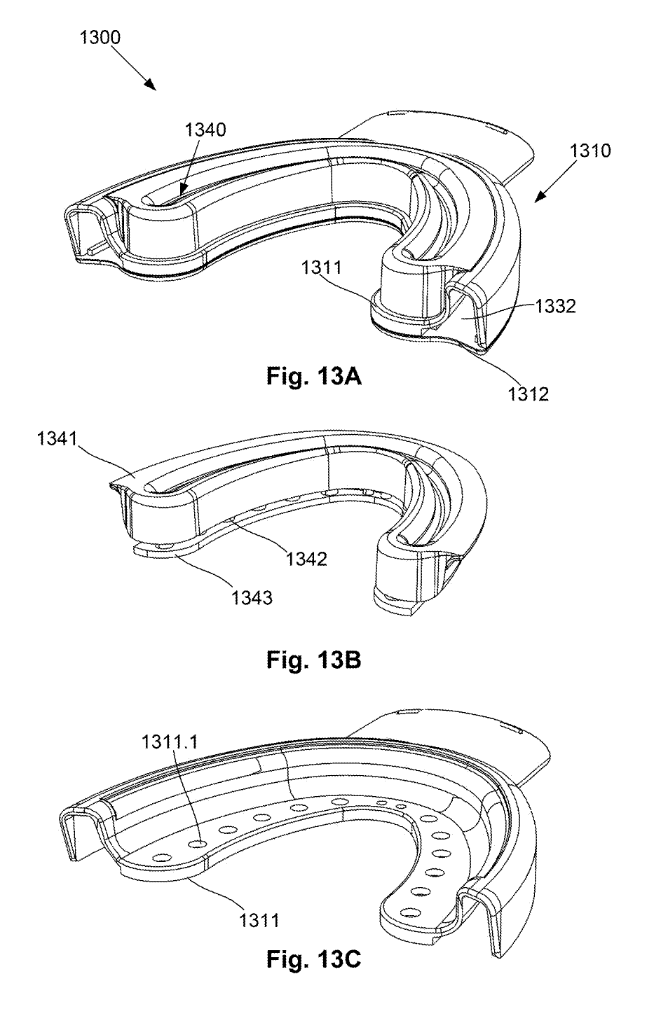

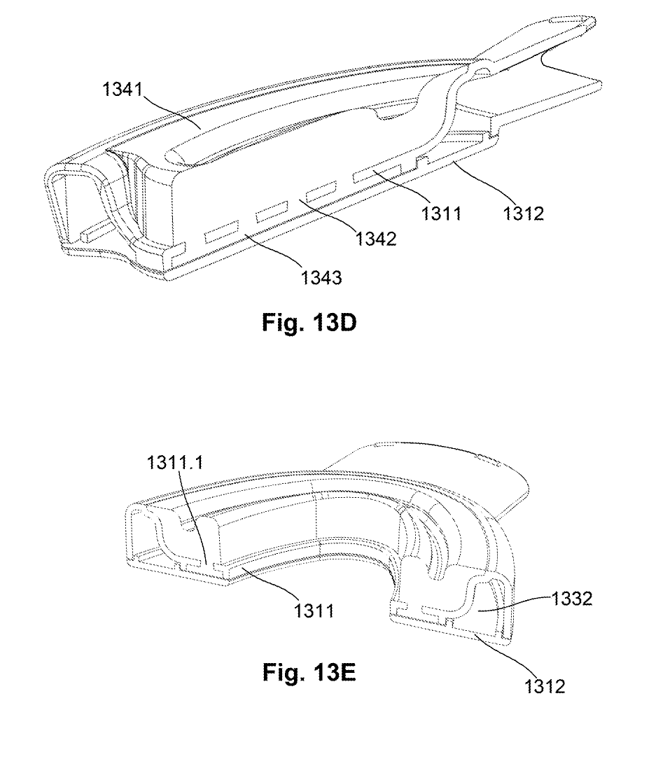

An alternative example of a first body and insert will now be described with reference to FIGS. 13A to 13E.

In this example, the first body includes a base 1312 and cover 1311 in a manner similar to that described above with respect to FIGS. 11A to 11E. In this example, the cover 1311 includes a number of holes 1311.1 provided in a lower surface of the recess. The insert 1340 includes an insert body 1341 coupled to an insert base 1343 via a number of pillars 1342. In use, the pillars 1342 are adapted to extend through the holes 1311.1, so that the insert base 1343 is provided on an underside of the body between the base 1312 and cover 1311, thereby securing the insert in position.

In practice this configuration can be achieved by moulding the insert 1340 in situ, so that the silicone or other material passes through the holes 1311.1 into the region between the base 1312 and cover 1311, before setting, to thereby secure the insert in position. Alternatively, this can be achieved by having the insert base 1343 couple to the insert body 1341, for example by having the pillars couple to the insert body 1341 so that the pillars 1342 and insert body 1341 mechanically engage, either through an interference fit, adhesive attachment or the like. In this instance, the insert body 1341 is positioned in the recess of the cover 1311, with the insert base being positioned on the underside of the cover 1311, thereby fixing the insert into position. Following this, the base 1312 can be attached to form the first body 1310, with the insert 1340 coupled thereto.

It will be appreciated that a similar fixing arrangement can be used for other arrangements of first body.

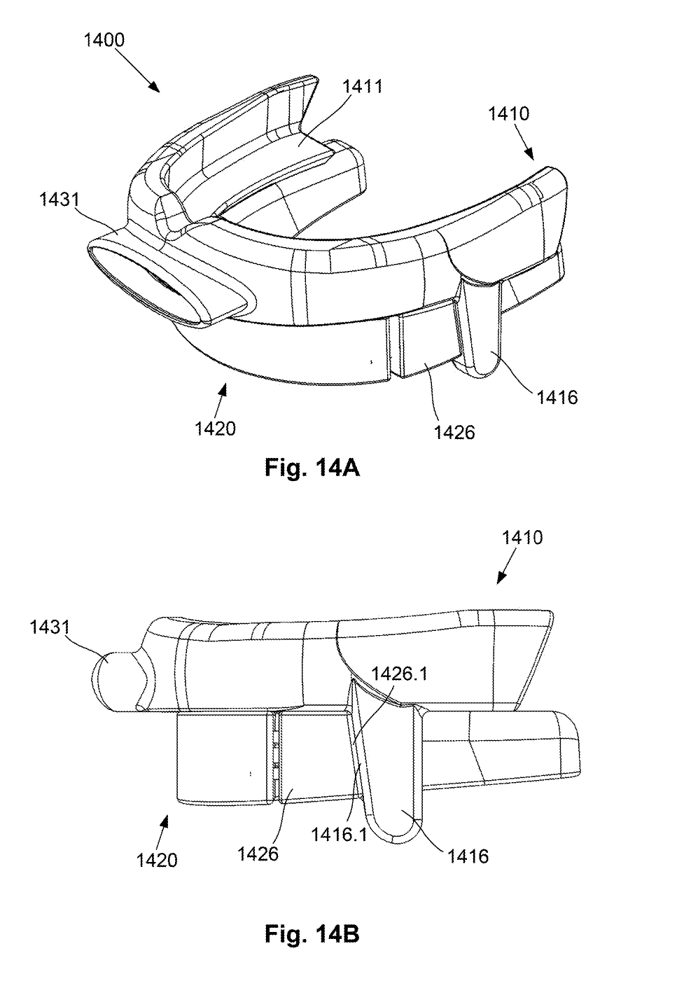

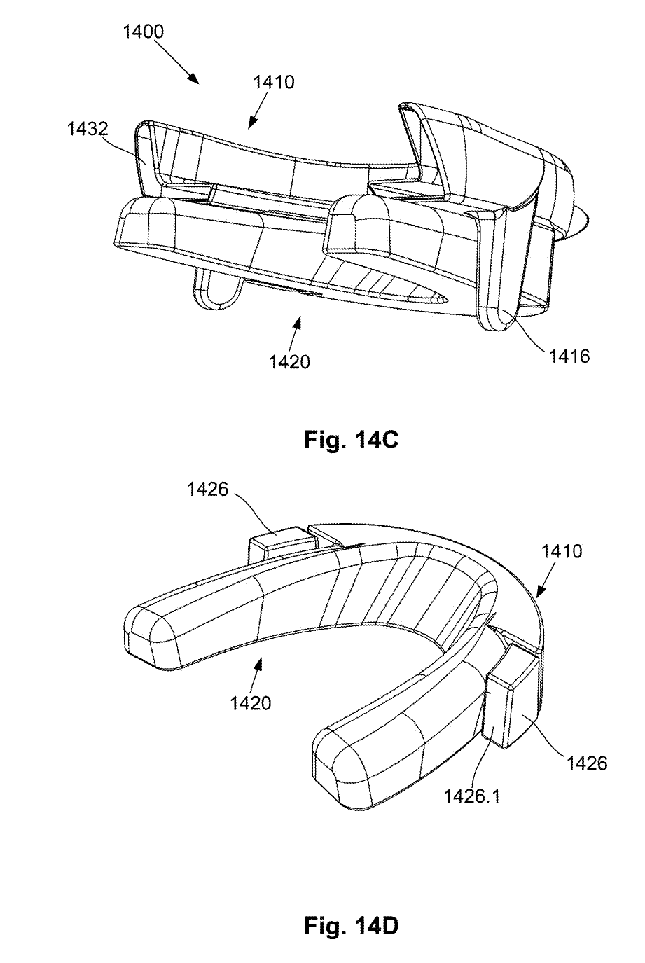

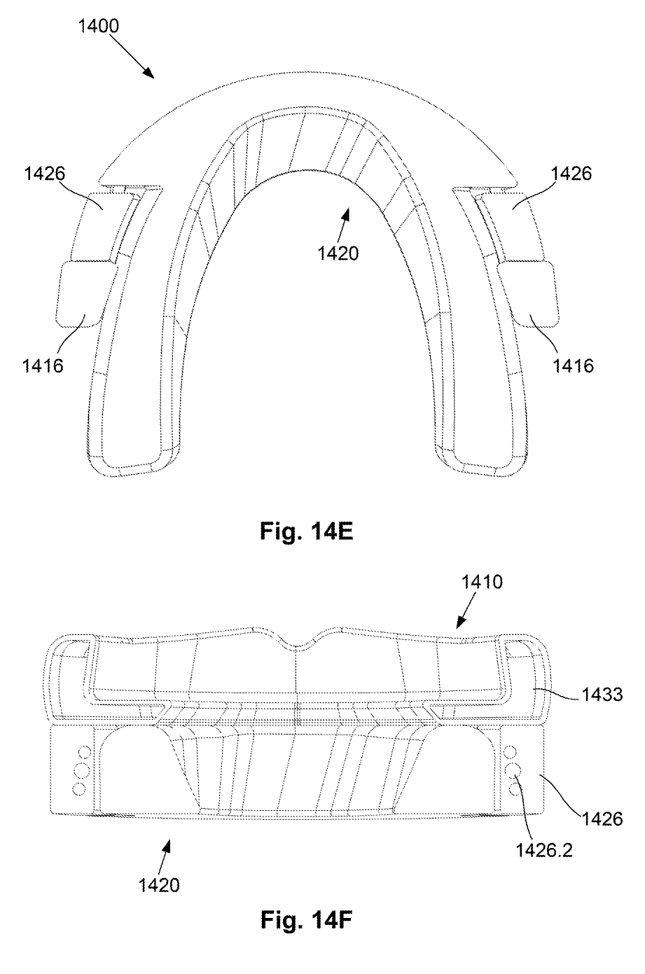

A further example of a breathing apparatus having an adjustment mechanism will now be described with reference to FIGS. 14A to 14F.

In this example, the apparatus 1400 again includes first and second bodies 1410, 1420, with a channel (not shown) extending from a first opening 1431 in a front of the first body to second openings 1432 in a rear of the first body, to allow airflow therethrough.

The first body 1410 includes wings 1416 extending downwardly from the first body so that these extend towards the second body in use. In one specific example, the wings are positioned mid-way along either side of the first body 1410, and are spaced to allow these to pass over side walls of the second body.

The second body also includes blocks 1426 adjustably mounted to the second body, and in particular movably mounted to outer side walls of the second body. In one example, this is achieved by having a front portion of the side wall extend outwardly to define a shoulder, with the blocks being attached to a rear surface of the shoulder by screws or other similar fasteners provided in holes 1426.2 passing through the block. Adjustment of the screws allows the blocks 1426 to be moved in a longitudinal direction.

In use a front surface 1416.1 of the wings 1416 engage a rearward surface 1426.1 of the blocks 1426 to constrain relative longitudinal movement of the first and second bodies 1410, 1420, in particular preventing the second body moving rearwardly relative to the first body 1410. This in turn, through appropriate positioning of the blocks 1426, can be used to selectively advance the user's mandibular teeth relative to the maxillary teeth, thereby providing mandibular advancement.

In one specific example, the forward surface 1416.1 of the wings 1416 and a rearward surface 1426.1 of the block 1426 are sloped to thereby urge the second body in a longitudinal forward direction as the first and second bodies are brought into engagement, thereby assisting with moving the mandible forwards as the user closes their jaws.

It will be appreciated that in this instance, as each block can be independently adjusted, this allows mandibular repositioning to be performed on each side of the jaw as needed. It will also be appreciated that the length of the wings could be adjusted depending on the requirements of the user.

It will also be appreciated that the use of blocks, whilst desirable, is not essential, and alternatively the wing could be adjustable mounted to the shoulder directly, for example using screws configured to sit within channel in the wings, thereby allowing the wings to move in an up and down direction, whilst preventing forward or rearward movement between the first and second bodies.

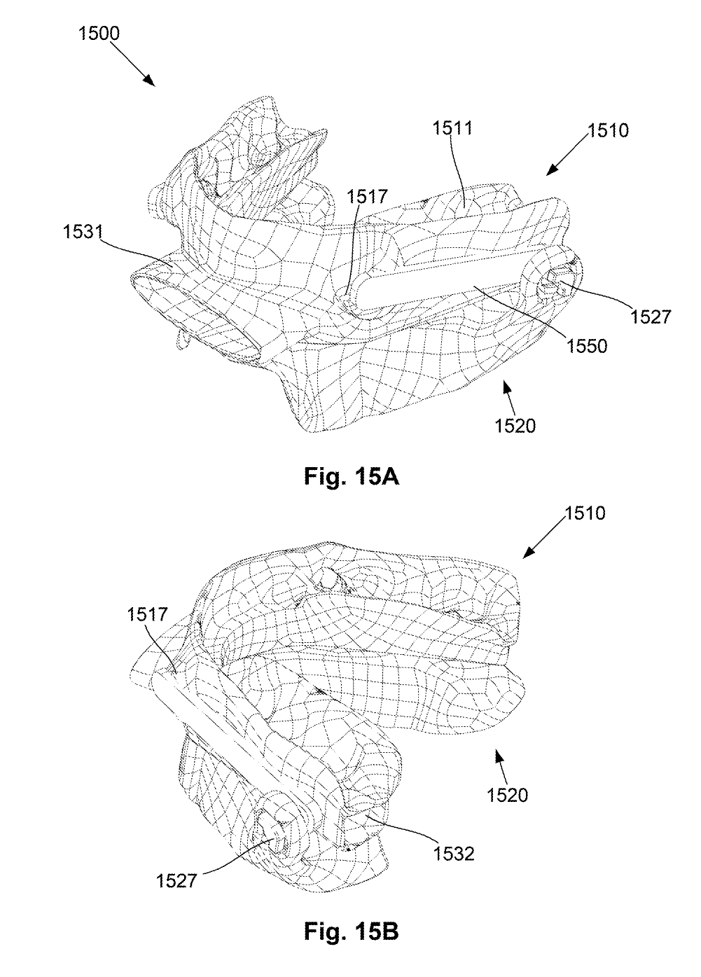

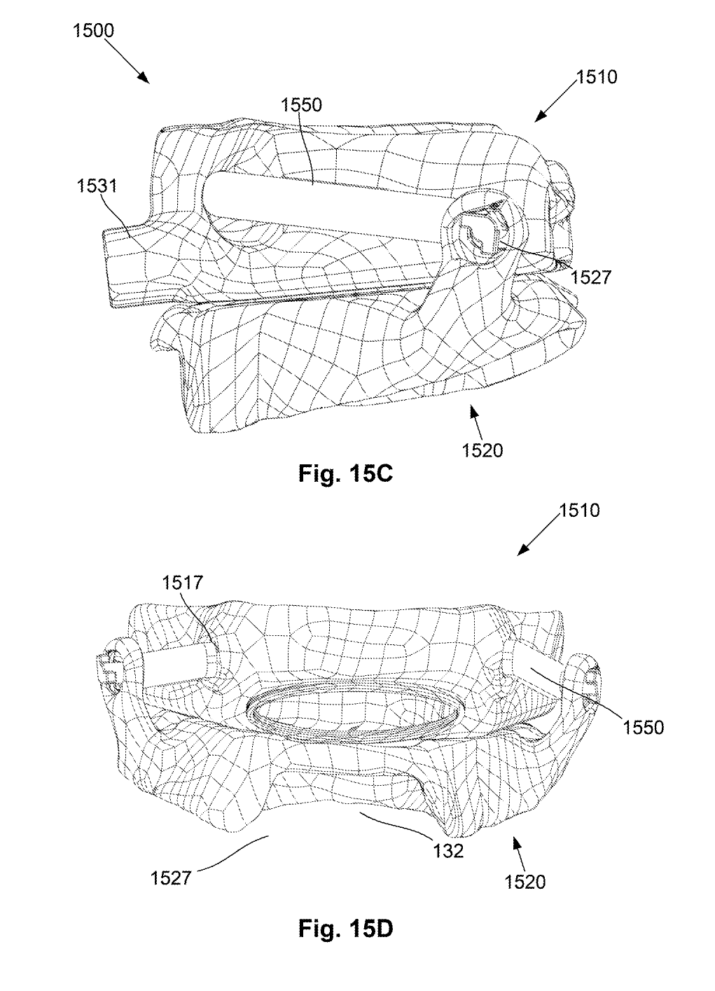

A further example of a breathing apparatus having an adjustment mechanism will now be described with reference to FIGS. 15A to 15D.

In this example, the apparatus 1500 again includes first and second bodies 1510, 1520, with a channel (not shown) extending from a first opening 1531 in a front of the first body to second openings 1532 in a rear of the first body, to allow airflow therethrough.

In this example, the apparatus is manufactured using laser sintering of a nylon material, and includes the inserts integrally formed with the first and second bodies, so that the user positions their teeth directly in the recesses 1511, 1521 of the first and second bodies 1510, 1520. It will be appreciated from this, that the apparatus is manufactured based according to customised specifications for a particular user.

In this example, the apparatus is designed to allow a degree of mandibular advancement to be fixed for the user, through the use of arms 1550 mounted to a fore side wall of the first body 1510 and a rear side wall of the second body 1520. Specifically, the arms are pivotally mounted via pivotal attachment points 1517, 1527, allowing the user to open their jaws with the apparatus in position, whilst retaining the desired degree of mandibular advancement. It will be appreciated that as the apparatus is manufactured for a specific user, adjustment of the degree of mandibular advancement might not be required.

However, in the event that adjustment is desired, this could be achieved by adjusting a length of the arms, for example by removing and replacing the arms 1550 with arms of a different length. Alternatively, either of the adjustment points could be configured to allow a pivot point to be moved, to thereby adjust the degree of advancement.

It will be appreciated that the overall configuration of the apparatus described above, when the first and second bodies are combined, is similar to arrangements described in copending applications PCT/AU2012/000565 and PCT/AU2015/050144, the contents of which are incorporated herein by reference.

For example, the apparatus can include a filter 115 for filtering air flowing through the apparatus. This can help remove particulates, pollen or other contaminants entrained in air flowing into the device, which can assist in reducing respiratory irritation, which can in turn exacerbate snoring and breathing difficulties. The filter 115 can be positioned anywhere within the body 110, but is typically provided within the first opening 131, thereby allowing this to be easily removed and replaced if required. The filter could be of any suitable form and could include a porous plastic or cloth based filter, and may include additional materials for added functionality. For example, the filter can also include activated carbon for filtering out pollution/bacteria.

Additionally, and/or alternatively a heat/moisture exchanger can be provided that controls the water and temperature content of the air being inhaled by exchanging heat and moisture with exhaled air. Examples of such exchangers can be found for example in U.S. Pat. No. 5,433,192, and these will not therefore be described in any further detail.

Additionally or alternatively, the apparatus can include a valve (not shown) for regulating air flow through the apparatus. In one example, this can be used to resist outflow of air from the second openings to the first opening. This can assist in regulating breathing and in particular allow for rapid inhalation, whilst ensuring slower exhalation, thereby optimising gas exchange within the lungs, for example to minimise the chances of hyperventilation. The valve can be of any suitable form, such as a ball valve, umbrella valve, or the like, and can be adjustable or titratable to ensure that the level of flow control is appropriate to the user.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers.

Persons skilled in the art will appreciate that numerous variations and modifications will become apparent. All such variations and modifications which become apparent to persons skilled in the art, should be considered to fall within the spirit and scope that the invention broadly appearing before described.

1) Apparatus for providing breathing assistance, the apparatus including:
a) first and second bodies configured to be positioned within an oral cavity of a user, each of the first and second bodies including a recess configured to receive a respective set of teeth of a user in use; and,
b) an adjustable mounting configured to interconnect the first and second bodies to thereby allow a relative position of the first and second bodies to be adjusted in a longitudinal direction to thereby selectively advance the mandibular teeth relative to the maxillary teeth, wherein at least one of the first and second bodies includes:
i) at least one first opening for allowing airflow between lips of the user;
ii) at least one second opening provided in the oral cavity to allow air flow into and out of a posterior region of the oral cavity; and,
iii) at least one channel connecting the at least one first and second openings.

2)-4) (canceled)

5) Apparatus according to claim 1, wherein the adjustable mounting includes:
a) a threaded shaft rotatably mounted to the first body, the shaft extending in a longitudinal direction;
b) a threaded carriage mounted on the threaded shaft to allow the carriage to move longitudinally in response to rotation of the shaft;
c) a peg coupled to the carriage; and,
d) a slot in the second body, the peg being adapted to engage the slot in the second body.

6)-10) (canceled)

11) Apparatus according to claim 5, wherein the threaded shaft is provided at least partially within the first opening and wherein the threaded shaft includes a plug at a first end, the plug being rotatably mounted within a socket in the first body and a head at a second end, the head being adapted to allow rotation of the threaded shaft.

12) Apparatus according to claim 11, wherein at least one of:
a) the socket is defined by a socket component including:
i) a socket body incorporating the socket; and,
ii) a flange extending around at least part of one edge of the socket body, wherein the socket component is mounted in a recessed opening within the first body so that the flange engages the recess with a flange surface being provided substantially flush with an underside of the first body;
b) the first body includes a collar extending at least partially around the threaded shaft proximate the second end, with the head being recessed within the collar;
c) the first body includes a carriage slot, the carriage being at least partially accommodated within the carriage slot allowing a position of the carriage relative to the first body to be determined;
d) the first body includes visual indicia, allowing a relative position of the first and second bodies in a longitudinal direction, to be determined.

13)-15) (canceled)

16) Apparatus according to claim 1, wherein the adjustable mounting includes:
a) a first mounting member coupled to the first body;
b) a second mounting member coupled to the second body, the second mounting member being slidably mounted to the first mounting member; and,
c) a wheel rotatably mounted to one of the mounting members and adapted to engage the other mounting member to thereby cause relative movement of the mounting members upon rotation of the wheel to provide:
i) a visual indication of a relative position of the first and second mounting members; and,
ii) an actuator for controlling a relative position of the first and second bodies.

17)-22) (canceled)

23) Apparatus according to claim 1, wherein the adjustable mounting includes:
a) wings extending from the first body towards the second body in use;
b) blocks adjustably mounted to the second body to allow the blocks to be moved in a longitudinal direction and wherein in use the wings engage the blocks to constrain relative longitudinal movement of the first and second bodies to thereby selectively advance the mandibular teeth relative to the maxillary teeth and wherein a forward surface of the wings and a rearward surface of the block are sloped to thereby urge the second body in a longitudinal forward direction as the first and second bodies are brought into engagement.

24) (canceled)

25) Apparatus according to claim 1, wherein the adjustable mounting includes arms pivotally mounted to the first and second bodies to hold the bodies in a defined relative longitudinal position and wherein the mounting is adjusted by at least one of:
a) adjusting a length of the arms; and,
b) adjusting a pivot point.

26)-28) (canceled)

29) Apparatus according to claim 1, wherein each channel includes:
a) a first channel portion extending through the user's buccal cavity; and,
b) a second channel portion in fluid communication with the first channel portion and extending between the user's maxillary and mandibular teeth and wherein at least one of:
i) at least one of the cross sectional shape and cross sectional area of at least one of the first and second channel portions varies from the first opening to the second opening;
ii) a shape and size of the channels varies in accordance with an anatomy of the oral cavity of the user;
iii) each channel directs air through a hammular notch of the user; and,
iv) the second openings are angled inwardly at least one of:
(1) between 10° and 50°;
(2) between 20° and 40°; and,
(3) approximately 30°.

30)-34) (canceled)

35) Apparatus according to claim 1, wherein each body is at least one of:
a) made of at least one of:
i) nylon;
ii) metal;
iii) titanium alloys;
iv) high strength polymers; and,
v) cobalt chromium alloys;
b) is made using at least one of:
i) additive manufacturing;
ii) selective laser sintering; and,
iii) moulding; and,
c) is coated with at least one of:
i) a medical grade polymer;
ii) a medical grade elastomer;
iii) silicone;
iv) polyurethane;
v) epoxy; and,
(1) parylene.

36) (canceled)

37) Apparatus according to claim 1, wherein the apparatus is made by selecting one of a plurality of fixed size first bodies and one of a plurality of fixed size second bodies, based on a shape of jaws and teeth of the user.

38)-39) (canceled)

40) Apparatus according to claim 1, wherein the apparatus includes at least one insert positioned in a recess of one of the first and second bodies, the insert being positioned at least partially between the user's teeth and the respective body in use and wherein the insert is at least one of:
a) customised for a user's teeth;
b) at least one of removable and replaceable;
c) a temporary insert that is at least one of:
i) a moulded insert;
ii) a silicone moulded insert; and,
iii) created at least partially in situ; and,
d) a semi-permanent insert that is at least one of:
i) a moulded insert;
ii) an acrylic insert; and,
iii) created at least in part using additive manufacturing; and,
e) made of at least one of:
i) metals;
ii) ceramics;
iii) a polymer;
iv) polyvinylsiloxane;
v) polyurethane; and,
vi) ethylvinylacetate.

41)-47) (canceled)

48) Apparatus according to claim 40, wherein the apparatus includes at least one insert positioned in a recess of one of the first and second bodies, the insert being positioned at least partially between the user's teeth and the respective body in use and wherein at least one of
a) each insert includes a base attached to the insert by a number of pillars and wherein, in use, the base is positioned on an underside of the body, with the pillars extending through apertures in the body to thereby secure the insert to the body;
b) each recess includes spaced apart recess side walls extending from a recess base, and wherein each insert includes an insert body shaped to at least partially conform to the recess side walls and recess base of a respective recess; and,
c) at least one recess side wall includes a recess lip projecting inwardly from at least part of the side wall, and wherein the recess lip is adapted to engage at least part of the insert body to thereby at least partially retain the insert body within the recess.

49)-53) (canceled)

54) Apparatus according to claim 1, wherein the first body includes a base and a cover coupled to the base, the base and cover defining:
a) at least one first opening for allowing airflow between lips of the user;
b) at least one second opening provided in the oral cavity to allow air flow into and out of a posterior region of the oral cavity; and,
c) at least one channel connecting the at least one first and second openings.

55) Apparatus for providing breathing assistance, the appliance including:
a) first and second bodies configured to be positioned within an oral cavity of a user, each of the first and second bodies including a recess having spaced apart recess side walls extending from a recess base and a recess lip projecting inwardly from at least part of at least one side wall; and,
b) first and second inserts, each insert having an insert body shaped to at least partially conform to the recess side walls and recess base of a respective recess, the insert body and recess lip engaging to thereby retain the insert within the respective recess, and the insert body being configured to receive a respective set of teeth of a user in use.

56)-66) (canceled)

67) Apparatus for providing breathing assistance, the apparatus including:
a) a first body including a base and a cover coupled to the base, the base and cover define:
i) at least one first opening for allowing airflow between lips of the user;
ii) at least one second opening provided in the oral cavity to allow air flow into and out of a posterior region of the oral cavity; and,
iii) at least one channel connecting the at least one first and second openings;
b) a second body configured to be positioned within an oral cavity of a user; and,
c) first and second inserts, each being coupled to a respective one of the first and second bodies, each insert being positioned at least partially between the user's teeth and the respective body in use, and the inserts being moulded to fit the user's teeth.

68) Apparatus according to claim 67, wherein the second openings are at least one of:
a) angled inwardly at least one of:
i) between 10° and 50°;
ii) between 20° and 40°; and,
iii) approximately 30°;
b) positioned over a last or back tooth on each side of the top jaw; and,
c) configured to direct air through a hammular notch of the user.

69) Apparatus according to claim 67, wherein each body is made of an injection moulded polymer that is at least one of:
a) a thermosetting polymer;
b) a thermoplastic polymer;
c) silicone;
d) an elastomer;
e) polycarbonate;
f) acrylonitrile butadiene styrene;
g) polyvinylsiloxane;
h) polyurethane; and,
i) ethylvinylacetate.

70) Apparatus according to claim 67, wherein each insert is made of at least one of:
a) an injection moulded polymer;
b) an additive printed material; and,
c) a deformable material set in the shape of the user's teeth.

71) Apparatus according to claim 67, wherein the first body includes an adjustable mounting configured to interconnect the first and second bodies to thereby allow a relative position of the first and second bodies to be adjusted.

72) Apparatus according to claim 71, wherein the adjustable mounting is configured to adjust a relative position of the first and second bodies in a longitudinal direction aligned with a dental midline of the user in use to thereby selectively advance the mandibular teeth relative to the maxillary teeth.

73)-75) (canceled)
